(19) United States
(12) Patent Application Publication
(10) Pub. No.: US 2023/0174581 A1
(43) Pub. Date: Jun. 8, 2023

(54) PROCESSES AND AGENTS FOR GLAUCOMA

(71) Applicant: AUFBAU MEDICAL INNOVATIONS LIMITED, Dublin (IE)

(72) Inventors: John T. G. PENA, New York, NY (US); James Murray MITCHELL, Danville, CA (US); Melissa A. MORGAN, Ithaca, NY (US); Harmon Lawrence REMMEL, New York, NY (US)

(73) Assignee: AUFBAU MEDICAL INNOVATIONS LIMITED, Dublin (IE)

(21) Appl. No.: 17/934,661

(22) Filed: Sep. 23, 2022

Related U.S. Application Data
(63) Continuation of application No. PCT/US2021/023825, filed on Mar. 24, 2021.
(60) Provisional application No. 62/994,469, filed on Mar. 25, 2020.

(51) Int. Cl. C07K 7/58

(52) U.S. Cl. CPC C07K 7/58

(57) ABSTRACT

This invention relates to methods and compositions for treating diseases of intraocular pressure. More particularly, this invention discloses a range of compounds, devices and methods for detecting and/or affecting intraocular pressure, treating glaucoma diseases, and increasing ocular outflows. Compositions of this disclosure can be used for reducing ocular extracellular complexes.

### TECHNICAL FIELD

This invention relates to methods, compounds and compositions for use in treating glaucoma diseases. More particularly, this invention discloses compositions and methods for affecting intraocular pressure, increasing ocular outflows, and/or reducing ocular extracellular features in glaucoma.

### SEQUENCE LISTING

This application contains a Sequence Listing submitted electronically in ASCII format created Mar. 16, 2021, named 12307_003WO1_SL.txt, which is 725 bytes in size and is hereby incorporated by reference.

### BACKGROUND

Glaucoma diseases are a world-wide leading cause of vision loss and affect an estimated 70 million people. Glaucoma is a permanently blinding disease.

Elevated intraocular pressure (IOP) or ocular hypertension is a risk factor for glaucoma. Human eye pressure greater than 22 mm Hg is considered higher than normal, which ranges from 12-22 mm Hg. Signs and symptoms of glaucoma include damage to the optic nerve, with degeneration of retinal ganglion cells, changes to the optic nerve head, and corresponding visual field loss. Elevation of IOP is a major risk factor related to retinal ganglion cell (RGC) death and ultimately visual field (VF) loss.

Elevated IOP is a significant risk factor for the progression from ocular hypertension to glaucoma disease, and is the only common clinical finding in a wide variety of secondary glaucomas. Forms of glaucoma are described as open angle glaucoma or closed angle glaucoma. Primary open-angle glaucoma (POAG) is most prevalent at about 75% of cases. In POAG, there is elevated intraocular pressure with no underlying disease.

Elevated IOP may be caused by aggregation of extracellular features in aqueous ocular humor into complexes or bodies which reduce ocular outflows and increase IOP.

Pharmaceutical treatment of glaucoma is directed to reducing IOP. Drawbacks of current treatments include lack of efficacy in reducing IOP, inability to reduce formation of extracellular bodies or complexes in aqueous ocular humor, and side effects of medications.

Further drawbacks in the field include the use of conventional tonometers for measuring ocular pressure. These devices generally lack accuracy and precision for measuring IOP. Because of these drawbacks, there are severe limitations in measuring IOP and treating glaucoma by reducing IOP in patients.

What is needed are effective methods, compounds and compositions for glaucoma, as well as modalities for reducing IOP and improving ocular outflows.

There is an urgent need for methods, devices, and compositions for reducing IOP, reducing formation of ocular extracellular matrix bodies and/or complexes, as well as for treating glaucoma diseases.

### BRIEF SUMMARY

This invention provides methods, compositions, and devices for IOP and glaucoma, including modalities for reducing IOP, improving ocular outflows, reducing formation of ocular extracellular matrix bodies and/or complexes, as well as for treating glaucoma diseases.

In some aspects, this invention provides methods, compounds and compositions for reducing intraocular pressure and increasing ocular outflows in glaucoma subjects. Aspects of this invention can reduce formation and presence of extracellular features and structures in ocular humor.

In further aspects, this disclosure provides therapeutic compounds and compositions for treating glaucoma.

Embodiments of this invention also provide devices for measuring and characterizing the effects of glaucoma extracellular features, as well as testing agents for activity in reducing intraocular pressure (IOP).

Embodiments of this invention include the following:

A pharmaceutical composition for ophthalmic use comprising a cyclic peptide active agent. The cyclic peptide may be a cyclic hepapeptide with a tripeptide side branch.

The composition above, wherein the active agent has Formula XV

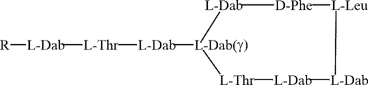

wherein R is selected from alkyl, cycloalkyl, aminoalkyl, alkenyl, alkynyl, alkanoyl, alkenoyl, wherein Dab is a diaminobutanoic acid monomer, and pharmaceutically-acceptable prodrugs, esters and salts thereof. R may be 6-methyloctanoyl (B₁), 6-methylheptanoyl (B₂), octanoyl (B₃), heptanoyl (B₄), and pharmaceutically-acceptable prodrugs, esters and salts thereof. R may be selected from alkyl, cycloalkyl, aminoalkyl, alkenyl, alkynyl, alkanoyl, alkenoyl; and pharmaceutically-acceptable prodrugs, esters and salts thereof; preferably excluding polymyxin, polymyxin B for use in treating glaucoma; more preferably excluding polymyxin, polymyxin B and all pharmaceutically acceptable prodrugs, esters and salts thereof for use in treating glaucoma; even more preferably excluding polymyxin, polymyxin B and all pharmaceutically acceptable prodrugs, esters and salts thereof for any use.

The composition above, wherein the active agent has Formula XVI

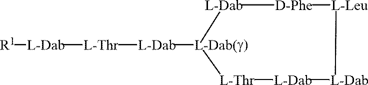

wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy; and pharmaceutically-acceptable prodrugs, esters and salts thereof.

In some embodiments, R¹ may be a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy; and pharmaceutically-acceptable prodrugs, esters and salts thereof; preferably excluding polymyxin, polymyxin B for use in treating glaucoma; more preferably excluding polymyxin, polymyxin B and all pharmaceutically acceptable prodrugs, esters and salts thereof for use in treating glaucoma; even more preferably excluding polymyxin, polymyxin B and all pharmaceutically acceptable prodrugs, esters and salts thereof for any use.

In certain embodiments, R¹ may be a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In further embodiments, R¹ may be a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof; preferably excluding polymyxin, polymyxin B for use in treating glaucoma; more preferably excluding polymyxin, polymyxin B and all pharmaceutically acceptable prodrugs, esters and salts thereof for use in treating glaucoma; even more preferably excluding polymyxin, polymyxin B and all pharmaceutically acceptable prodrugs, esters and salts thereof for any use.

The composition above, wherein the active agent has Formula XVII

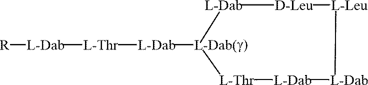

wherein R is selected from alkyl, cycloalkyl, aminoalkyl, alkenyl, alkynyl, alkanoyl, alkenoyl, and pharmaceutically-acceptable prodrugs, esters and salts thereof. R can be selected from alkyl, cycloalkyl, aminoalkyl, alkenyl, alkynyl, alkanoyl, alkenoyl, and pharmaceutically-acceptable prodrugs, esters and salts thereof.

The composition above, wherein the active agent has Formula XVIII

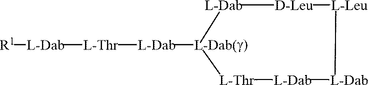

wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, R¹ may be a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In certain embodiments, R¹ may be a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In further embodiments, R¹ can be a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, R¹ may be a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof. R¹ can be a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

The active agent may have Formula XIX [0037]

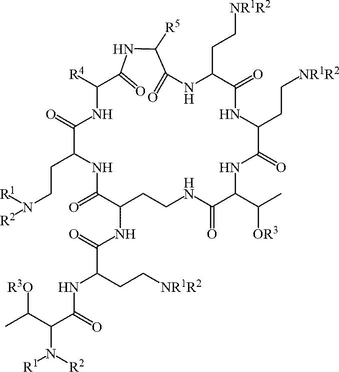

wherein
R¹, R² are independently selected from H, alkyl, cycloalkyl aminoalkyl, hydroxyalkyl, carboxylalkyl, aryl;
R³ is selected from H, alkyl, aminoalkyl, hydroxyalkyl, carboxylalkyl;
R⁴ is selected from H, alkyl, cycloalkyl, aminoalkyl, hydroxyalkyl, carboxylalkyl, benzyl, aryl, aralkyl, cycloalkyl-alkyl;
R⁵ is selected from H, alkyl, cycloalkyl, aminoalkyl, hydroxyalkyl, carboxylalkyl, aryl;
and pharmaceutically-acceptable prodrugs, esters and salts thereof.

The active agent may have Formula XX

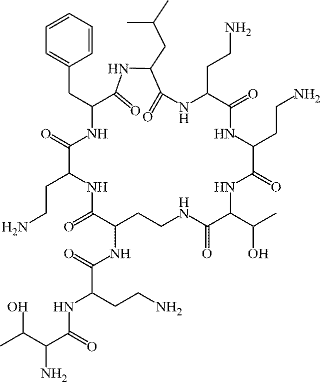 [SMILES: CC(C)CC1NC(=O)C(Cc2ccccc2)NC(=O)C(CCN)NC(=O)C(NC(=O)C(CCN)NC(=O)C(N)C(C)O)CCNC(=O)C(C(C)O)NC(=O)C(CCN)NC(=O)C(CCN)NC1=O]

and pharmaceutically-acceptable prodrugs, esters and salts thereof.

The active agent may have Formula XXI

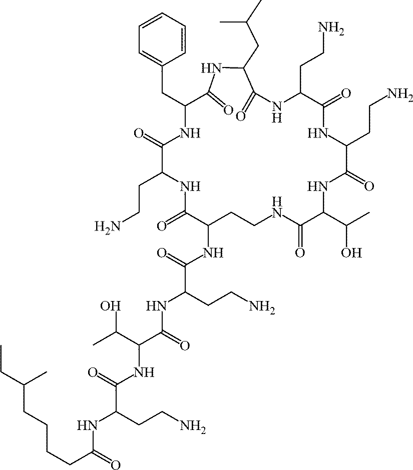 [SMILES: CCC(C)CCCCC(=O)NC(CCN)C(=O)NC(C(=O)NC(CCN)C(=O)NC1CCNC(=O)C(C(C)O)NC(=O)C(CCN)NC(=O)C(CCN)NC(=O)C(CC(C)C)NC(=O)C(Cc2ccccc2)NC(=O)C(CCN)NC1=O)C(C)O]

and pharmaceutically-acceptable prodrugs, esters and salts thereof.

The composition above, wherein the active agent has Formula XXII

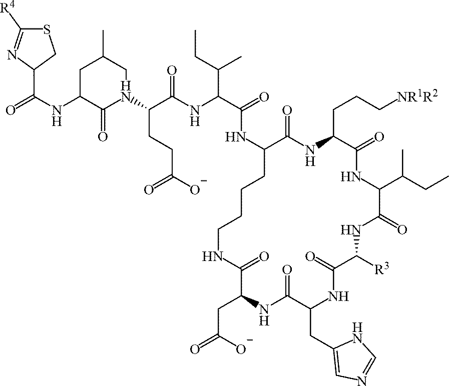

wherein R¹, R² are independently selected from H, alkyl, cycloalkyl, aminoalkyl, hydroxyalkyl, carboxylalkyl, aryl;
R³ is selected from H, alkyl, cycloalkyl, aryl, benzyl, arylalkyl;
R⁴ is selected from H, alkyl, cycloalkyl, aryl, aminoalkyl, arylalkyl;
and pharmaceutically-acceptable prodrugs, esters and salts thereof.

The composition above, wherein the active agent is bacitracin A, and pharmaceutically-acceptable prodrugs, esters and salts thereof.

A pharmaceutical composition for ophthalmic use comprising a pyridinium active agent. The active agent may have Formula X

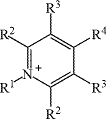

wherein
R¹ is selected from alkyl, cycloalkyl, aminoalkyl, acylalkyl, benzyl, alkenyl, alkynyl, wherein R¹ is terminated with H, a carbon-carbon double bond, or a methacryloyloxy group;
R², R³ are independently selected from H, halo, alkyl;
R⁴ is selected from H, OH, alkoxy, alkylalkoxy, aminoalkoxy, hydroxyalkoxy, carboxyalkoxy, haloalkoxy, alkoxyalkoxy, benzyl, amino, alkylamino, cycloalkylamino, carboxyalkylamino, carboxylate-alkylamino; and pharmaceutically-acceptable prodrugs, esters and salts thereof; preferably excluding cetylpyridinium for use in treating glaucoma; more preferably excluding cetylpyridinium and all pharmaceutically acceptable prodrugs, esters and salts thereof for use in treating glaucoma; even more preferably excluding cetylpyridinium and all pharmaceutically acceptable prodrugs, esters and salts thereof for any use.

In some embodiments, R¹ can be selected from alkyl, cycloalkyl, aminoalkyl, alkenyl, alkynyl, wherein R¹ is terminated with H, a carbon-carbon double bond, or a methacryloyloxy group;
R², R³ are independently selected from H, halo, alkyl;
R⁴ is selected from H, OH, alkoxy, amino, alkylamino, cycloalkylamino; and pharmaceutically-acceptable prodrugs, esters and salts thereof; preferably excluding cetylpyridinium for use in treating glaucoma; more preferably excluding cetylpyridinium and all pharmaceutically acceptable prodrugs, esters and salts thereof for use in treating glaucoma; even more preferably excluding cetylpyridinium and all pharmaceutically acceptable prodrugs, esters and salts thereof for any use.

In certain embodiments, R¹ may be C(14-24)alkyl, C(14-24)alkenyl;
R², R³ are independently selected from H, halo, alkyl;
R⁴ is selected from H, OH, alkoxy, amino, alkylamino, cycloalkylamino; and pharmaceutically-acceptable prodrugs, esters and salts thereof; preferably excluding cetylpyridinium for use in treating glaucoma; more preferably excluding cetylpyridinium and all pharmaceutically acceptable prodrugs, esters and salts thereof for use in treating glaucoma; even more preferably excluding cetylpyridinium and all pharmaceutically acceptable prodrugs, esters and salts thereof for any use.

In some embodiments, R¹ can be C(14-24)alkenyl;
R², R³ are independently selected from H, halo, alkyl;
R⁴ is selected from H, OH, alkoxy, amino, alkylamino, cycloalkylamino; and pharmaceutically-acceptable prodrugs, esters and salts thereof.

In certain embodiments, the active agent can be C(16-18)alkyl-pyridin-1-ium, C(18:1(9))alkenyl-pyridin-1-ium, C(18:2(9,12))alkenyl-pyridin-1-ium, C(18:3(9,12,15))alkenyl-pyridin-1-ium, and pharmaceutically-acceptable prodrugs, esters and salts thereof.

A pharmaceutical composition for ophthalmic use comprising a peptidic active agent. The active agent for use in treating glaucoma may be at least 75%, or 80%, or 85%, or 90%, or 95% identical to a reference polypeptide. The reference polypeptide can be bivalirudin, hirudin, or rapastinel.

In some embodiments, the active agent may have formula XXIII

H-{d}FPRPGGGGNGDFEEIPEEYL-OHFormula XXIII

and pharmaceutically-acceptable prodrugs, esters and salts thereof. In certain embodiments, further comprising 1-5 monomers independently selected from Lys, His, Arg, at the N-terminus or the C-terminus. In further embodiments, further comprising conservative replacement of 1-5 monomers.

In some embodiments, the active agent may have formula XXIV

### Seq Id No:1

H-NGDFEEIPEEYLA-OHFormula XXIV

and pharmaceutically-acceptable prodrugs, esters and salts thereof. The composition may further comprise 1-5 monomers independently selected from Lys, His, Arg, at the N-terminus or the C-terminus. The composition may further comprise conservative replacement of 1-5 monomers.

In further embodiments, the active agent may have formula XXV

### Seq Id No:2

H-TPPT-NH2Formula XXV

and pharmaceutically-acceptable prodrugs, esters and salts thereof. The composition may further comprise 1-5 monomers independently selected from Lys, His, Arg, at the N-terminus or the C-terminus.

In some embodiments, the active agent may have formula XXVI

H-TPXₐₐT-NH2Formula XXVI

wherein Xₐₐ is a Proline monomer substituted at the branch carbon, where the substituent can be H, and pharmaceutically-acceptable prodrugs, esters and salts thereof. The composition can further comprise 1-5 monomers independently selected from Lys, His, Arg, at the N-terminus or the C-terminus. The active agent may be rapastinel, apimostinel, and pharmaceutically-acceptable prodrugs, esters and salts thereof.

The active agent can have Formula XXVII

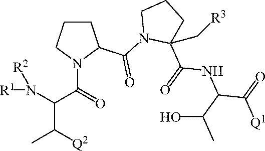

wherein
Q¹, Q² are independently selected from H, hydroxyl, amino, alkoxy, aryloxy, aminoalkoxy;
R¹, R² are independently selected from H, alkyl, cycloalkyl, aryl;
R³ is selected from H, alkyl, cycloalkyl aryl, haloalkyl, haloaryl, alkylaryl, haloalkylaryl;
and pharmaceutically-acceptable prodrugs, esters and salts thereof. The composition may further comprise 1-5 monomers independently selected from Lys, His, Arg, at the N-terminus or the C-terminus.

Embodiments of this invention include a pharmaceutical composition for ophthalmic use comprising a nucleoside phosphonate active agent. The active agent can have Formula I

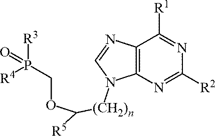

wherein
R¹ is selected from H, Cl, amino, alkylamino, cycloalkylamino, cycloalkyl-alkylamino, aminoalkylamino, carboxyalkylamino, benzylamino, =O;
R² is selected from H, Cl, ═O, NR⁶R⁷, alkylamino, cycloalkylamino, arylamino, benzylamino, 2-pyridinylamino, wherein R⁶, R⁷ are selected from H, alkyl, cycloalkyl, aryl;
R³,R⁴ are independently selected from OH, alkoxy, aminoalkoxy, hydroxyalkoxy, carboxyalkoxy, haloalkoxy, alkoxyalkoxy, carboxyalkylcarboxyl, carboxyalkenylcarboxyl, benzyloxy, amino, alkylamino, carboxyalkylamino, carboxylate-alkylamino, wherein R³,R⁴ may connect to form a loop;
R⁵ is selected from H, alkyl, cycloalkyl, hydroxyalkyl, aryl;
n is 1-5; and pharmaceutically-acceptable prodrugs, esters and salts thereof.

In some embodiments, the composition may have
R¹ is selected from H, Cl, amino, alkylamino, cycloalkylamino;
R² is selected from H, Cl, ═O;
R³, R⁴ are independently selected from OH, alkoxy, aminoalkoxy, hydroxyalkoxy, haloalkoxy, alkoxyalkoxy, amino, alkylamino, cycloalkylamino, wherein R³, R⁴ may connect to form a loop;
R⁵ is selected from H, alkyl, hydroxyalkyl;
n is 1-5; and pharmaceutically-acceptable prodrugs, esters and salts thereof.

The active agent may be adefovir, pradefovir, tenofovir, and pharmaceutically-acceptable prodrugs, esters and salts thereof.

The active agent may have Formula V

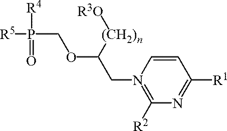

wherein
R¹ is selected from H, Cl, ═O, amino, alkylamino, cycloalkylamino, cycloalkyl-alkylamino, aminoalkylamino, carboxyalkylamino, benzylamino;
R² is selected from H, Cl, NR⁶R⁷, alkylamino, cycloalkylamino, arylamino, benzylamino, 2-pyridinylamino, wherein R⁶, R⁷ are selected from H, alkyl, cycloalkyl, aryl;
R³ is selected from H, alkyl, cycloalkyl, aryl;
R⁴, R⁵ are independently selected from OH, alkoxy, aminoalkoxy, hydroxyalkoxy, carboxyalkoxy, haloalkoxy, alkoxyalkoxy, carboxylalkenylcarboxyl, benzyloxy, amino, alkylamino, cycloalkylamino, carboxylalkylamino, carboxylate-alkylamino, wherein R³, R⁴ may connect to form a loop;
n is 1-5; and pharmaceutically-acceptable prodrugs, esters and salts thereof.

In certain embodiments, wherein
R¹ is selected from H, Cl, ═O, amino, alkylamino, cycloalkylamino;
R² is selected from H, Cl, amino, alkylamino, cycloalkylamino;
R³ is selected from H, alkyl, cycloalkyl, aryl;
R⁴, R⁵ are independently selected from OH, alkoxy, aminoalkoxy, hydroxyalkoxy, alkoxyalkoxy, benzyloxy, amino, alkylamino, cycloalkylamino;
n is 1-5; and pharmaceutically-acceptable prodrugs, esters and salts thereof.

The active agent can be cidofovir, brincidofovir, and pharmaceutically-acceptable prodrugs, esters and salts thereof.

The active agent may have Formula VIII

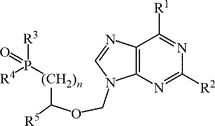

wherein
R¹ is selected from H, Cl, ═O, amino, alkylamino, cycloalkylamino, cycloalkyl-alkylamino, aminoalkylamino, carboxyalkylamino, benzylamino;
R² is selected from H, Cl, NR⁶R⁷, alkylamino, cycloalkylamino, arylamino, benzylamino, 2-pyridinylamino, wherein R⁶, R⁷ are selected from H, alkyl, cycloalkyl, aryl;
R³, R⁴ are independently selected from OH, alkoxy, aminoalkoxy, hydroxyalkoxy, carboxylalkoxy, haloalkoxy, alkoxyalkoxy, carboxylalkylcarboxyl, carboxylalkenylcarboxyl, benzyloxy, amino, alkylamino, cycloalkylamino, carboxylalkylamino, carboxylate-alkylamino, wherein R³, R⁴ may connect to form a loop;
R⁵ is selected from H, alkyl, hydroxyalkyl, aminoalkyl, aryl;
n is 1-5; and pharmaceutically-acceptable prodrugs, esters and salts thereof; preferably excluding acyclovir for use in treating glaucoma; more preferably excluding acyclovir and all pharmaceutically acceptable prodrugs, esters and salts thereof for use in treating glaucoma; even more preferably excluding acyclovir and all pharmaceutically acceptable prodrugs, esters and salts thereof for any use.

In some embodiments, wherein
R¹ is selected from H, Cl, ═O, amino, alkylamino, cycloalkylamino;
R² is selected from H, Cl, NR⁶R⁷, alkylamino, cycloalkylamino, wherein R⁶, R⁷ are selected from H, alkyl;
R³, R⁴ are independently selected from OH, alkoxy, aminoalkoxy, hydroxyalkoxy, haloalkoxy, alkoxyalkoxy, benzyloxy, amino, alkylamino, cycloalkylamino, wherein R³, R⁴ may connect to form a loop;
R⁵ is selected from H, alkyl, cycloalkyl, hydroxyalkyl, aryl;
n is 1-5; and pharmaceutically-acceptable prodrugs, esters and salts thereof; preferably excluding acyclovir for use in treating glaucoma; more preferably excluding acyclovir and all pharmaceutically acceptable prodrugs, esters and salts thereof for use in treating glaucoma; even more preferably excluding acyclovir and all pharmaceutically acceptable prodrugs, esters and salts thereof for any use.

The active agent may be acyclovir.

Embodiments of this invention include a pharmaceutical composition for ophthalmic use comprising a 9,10-dihydroanthracene active agent. The active agent may have Formula XXIX [00141]

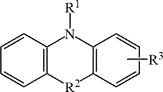

wherein
R¹ is selected from alkyl, cycloalkyl, aminoalkyl, hydroxyalkyl, alkoxyalkyl, aryl, alkenyl, amino-alkenyl, alkynyl, 1,4-piperazinyl, 1-alkyl-1,4-piperazinyl, 1-hydroxyalkyl-1,4-piperazinyl;
R² is selected from C, S, O;
R³ is selected from H, halo, alkyl, amino, —CF₃, —O—CH₃, —S—CH₃;
and pharmaceutically-acceptable prodrugs, esters and salts thereof.

The active agent may be chlorpromazine, fluphenazine, perphenazine, prochlorperazine, promethazine, thioridazine, phenothiazine, trifluoperazine, levomepromazine, chlorprothixene, and pharmaceutically-acceptable prodrugs, esters and salts thereof.

Additional embodiments of this invention include a pharmaceutical composition for ophthalmic use comprising a tripeptide active agent. The active agent can be boceprevir, levetiracetam, pramiracetam, and pharmaceutically-acceptable prodrugs, esters and salts thereof.

### BRIEF DESCRIPTION OF THE DRAWINGS

FIG. 1 shows that aqueous humor from a patient with primary open angle glaucoma increased the pressure in the microfluidic device. FIG. 1 shows the relative amount of pressure (mm Hg) change within an artificial trabecular meshwork formed by pillars in a microfluidic channel when infused with human aqueous humor obtained from a patient with severe primary open angle glaucoma. The fluid flow rate was held constant at 2 µl per minute, and the baseline system pressure was measured using an external pressure sensor. The human aqueous humor sample was injected at timepoint denoted by an arrow and the letter “a.” The pressure steadily rises to a maximum of about 41 mm Hg at 27 minutes. FIG. 1 shows that aqueous humor from patients diagnosed with POAG increased the pressure in the device.

FIG. 2 (top) shows a confocal photomicrograph of a microfluidic chip after capturing EMB from human aqueous humor from a patient with primary open angle glaucoma. Protein of the EMB was labeled with a fluorescent marker, carboxyfluorescein succinimidyl ester (CFSE, marked with arrows). The circles are pillars in a restriction channel. FIG. 2 (lower) shows EMB isolated in the microfluid channels around pillars.

FIG. 3 shows that agent colistin sulfate reduced intraocular pressure (IOP) in a human glaucoma model as compared to control. The agent was tested by controlling flow and measuring relative IOP using in a device of this invention. The agent was compared against placebo (buffered saline) by preparing each in aqueous humor from a patient with primary open angle glaucoma and pre-incubating at 37° C. for 24 hours. The timepoint of injection into the device is denoted by an arrow and the letter “a.” Referring to FIG. 25, the IOP for placebo (dashed line) increased greatly after injection of the placebo sample. The IOP rose to a maximum pressure of about 40 mm Hg. To the contrary, the IOP after injection of the agent colistin sulfate in human aqueous humor (solid line) was markedly lower than for placebo, up to about 40% lower, and the difference was sustained. This result showed that the agent colistin sulfate was surprisingly effective to reduce IOP in the human glaucoma model.

FIG. 4 shows the dose-response behavior of the compound colistin sulfate on reducing intraocular pressure (IOP) in a bovine vitreous glaucoma model.

FIG. 5 shows the dose-response behavior of the compound cetylpyridinium chloride on reducing intraocular pressure (IOP) in a bovine vitreous glaucoma model.

FIG. 6 shows the dose-response behavior of the compound polymyxin B sulfate on reducing intraocular pressure (IOP) in a bovine vitreous glaucoma model.

FIG. 7 shows the dose-response behavior of the compound rapastinel TFA on reducing intraocular pressure (IOP) in a bovine vitreous glaucoma model.

FIG. 8 shows the dose-response behavior of the compound adefovir on reducing intraocular pressure (IOP) in a bovine vitreous glaucoma model.

FIG. 9 shows the dose-response behavior of the compound levetiracetam on reducing intraocular pressure (IOP) in a bovine vitreous glaucoma model.

FIG. 10 shows the dose-response behavior of the compound chlorpromazine HCl on reducing intraocular pressure (IOP) in a bovine vitreous glaucoma model.

FIG. 11 shows the dose-response behavior of the compound boceprevir on reducing intraocular pressure (IOP) in a bovine vitreous glaucoma model.

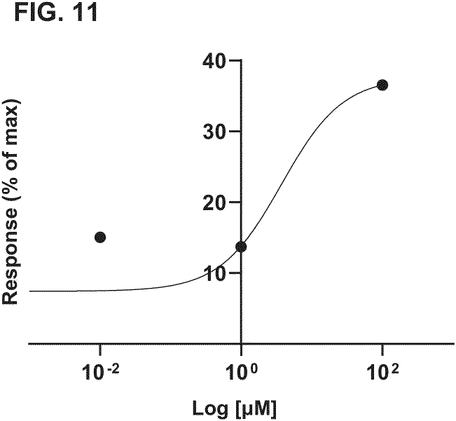

FIG. 12 shows that agent polymyxin B reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 12, the IOP for placebo (dashed line) increased greatly after injection of the placebo sample. The IOP rose steadily to a maximum pressure of about 250 mmHg. To the contrary, the IOP after injection of the agent polymyxin B (solid line) was 78% lower than for placebo, and the difference was sustained. This result showed that the agent polymyxin B was surprisingly effective to reduce IOP in the glaucoma model.

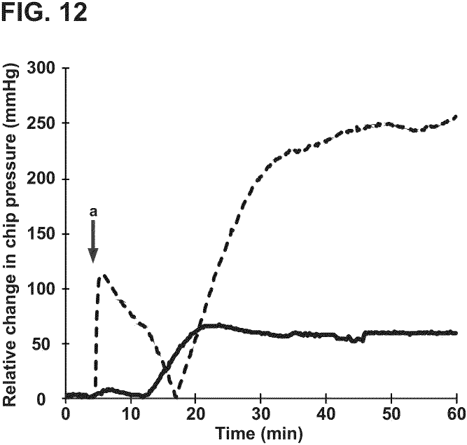

FIG. 13 shows that agent neomycin reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 13, the IOP for placebo (dashed line) increased greatly after injection of the placebo sample. The IOP rose steadily to a maximum pressure of about 64 mmHg. To the contrary, the IOP after injection of the agent neomycin (solid line) was 72% lower than for placebo, and the difference was sustained. This result showed that the agent neomycin was surprisingly effective to reduce IOP in the glaucoma model.

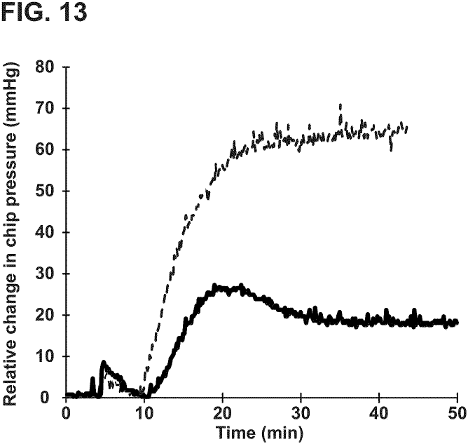

FIG. 14 shows that agent colistin sulfate reduced intraocular pressure (IOP) in a bovine glaucoma model as compared to control. The agent was tested by controlling flow and measuring relative IOP using in a device of this invention. The agent was compared against placebo (buffered saline) by preparing each in bovine aqueous humor (BVH) and pre-incubating at 37° C. for 24 hours. The timepoint of injection into the device is denoted by an arrow and the letter “a.” Referring to FIG. 14, the IOP for placebo (dashed line) increased greatly after injection of the placebo sample. The IOP rose to a maximum pressure of about 65 mm Hg. To the contrary, the IOP after injection of the agent colistin sulfate in BVH (solid line) was markedly lower than for placebo, up to about 97% lower, and the difference was sustained. This result showed that the agent colistin sulfate was surprisingly effective to reduce IOP in the glaucoma model.

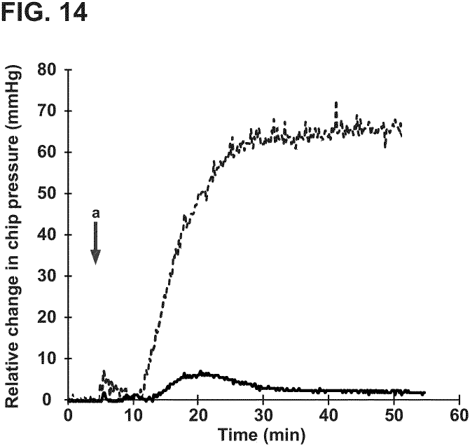

FIG. 15 shows that compound sodium dodecyl sulfate was a negative control for intraocular pressure (IOP) in a glaucoma model. The compound was tested by controlling flow and measuring relative IOP using in a device of this invention. The compound was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 15, the IOP for placebo (dashed line) increased greatly after injection of the placebo sample. The IOP rose steadily to a maximum pressure of about 60 mm Hg. However, the IOP after injection of sodium dodecyl sulfate (solid line) was significantly higher than for placebo. This result showed that sodium dodecyl sulfate was a negative control that did not reduce IOP in the glaucoma model.

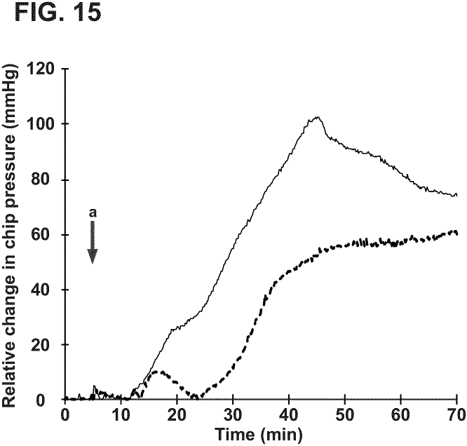

FIG. 16 shows that agent cetylpyridinium chloride reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 16, the IOP for placebo (dashed line) increased greatly after injection of the placebo sample. The IOP rose steadily to a maximum pressure of about 64 mm Hg. To the contrary, the IOP after injection of the agent cetylpyridinium chloride-BVH sample (solid line) was markedly lower than for placebo, up to nearly 100% lower, and the difference was sustained. This result showed that the agent cetylpyridinium chloride was surprisingly effective to reduce IOP in the glaucoma model.

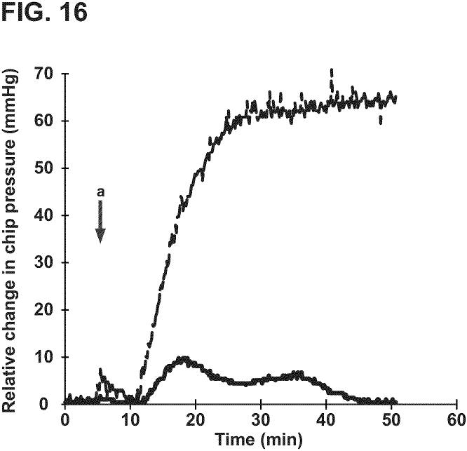

FIG. 17 shows that agent chlorpromazine reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 17, the IOP for placebo (dashed line) increased greatly after injection of the placebo sample. The IOP rose steadily to a maximum pressure of about 64 mm Hg. To the contrary, the IOP after injection of the agent chlorpromazine-BVH sample (solid line) was markedly lower than for placebo, up to about 81% lower, and the difference was sustained. This result showed that the agent chlorpromazine was surprisingly effective to reduce IOP in the glaucoma model.

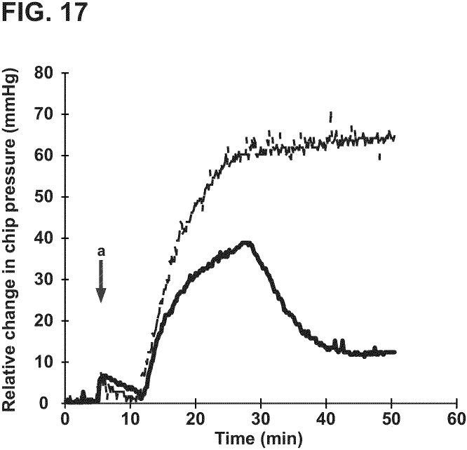

FIG. 18 shows that agent heparin sodium reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 18, the IOP for placebo (dashed line) increased greatly after injection of the placebo sample. The IOP rose steadily to a maximum pressure of about 67 mmHg. To the contrary, the IOP after injection of the agent heparin sodium (solid line) was 32% lower than for placebo, and the difference was sustained. This result showed that the agent heparin sodium was surprisingly effective to reduce IOP in the glaucoma model.

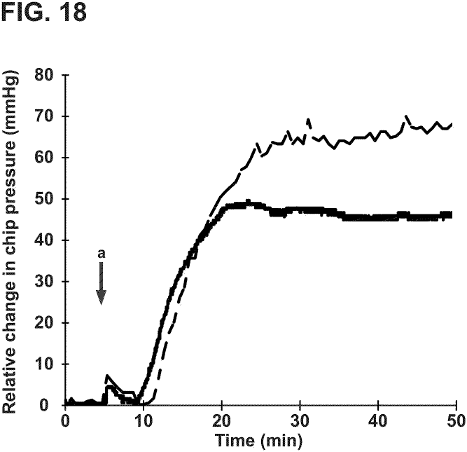

FIG. 19 shows that agent adefovir dipivoxil reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 19, the IOP for placebo (dashed line) increased greatly after injection of the placebo sample. The IOP rose steadily to a maximum pressure of about 112 mmHg. To the contrary, the IOP after injection of the agent adefovir dipivoxil (solid line) was up to 73% lower than for placebo, and the difference was sustained. This result showed that the agent adefovir dipivoxil was surprisingly effective to reduce IOP in the glaucoma model.

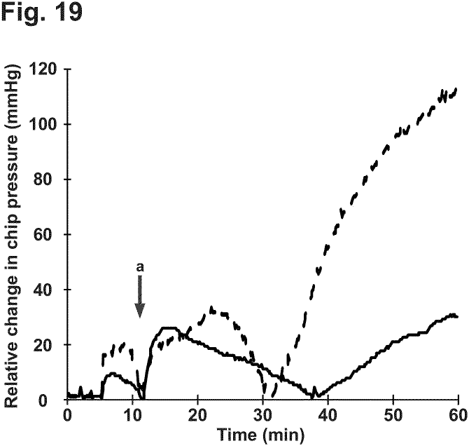

FIG. 20 shows that agent triflupromazine reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 20, the IOP for placebo (dashed line) increased greatly after injection of the placebo sample. The IOP rose steadily to a maximum pressure of about 112 mm Hg. To the contrary, the IOP after injection of the agent triflupromazine (solid line) was up to 40% lower than for placebo, and the difference was sustained. This result showed that the agent triflupromazine was surprisingly effective to reduce IOP in the glaucoma model.

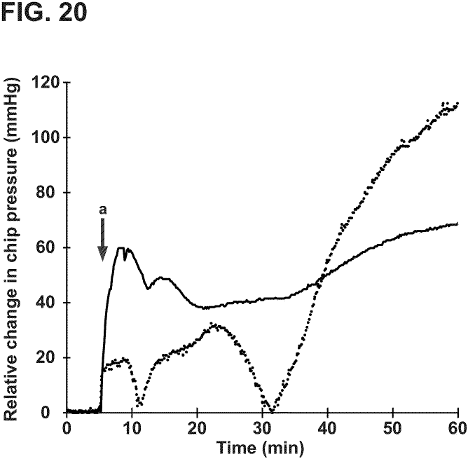

FIG. 21 shows that agent bacitracin zinc reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 21, the IOP for placebo (dashed line) increased greatly after injection of the placebo sample. The IOP rose steadily to a maximum pressure of about 113 mm Hg. To the contrary, the IOP after injection of the agent bacitracin zinc (solid line) was up to 58% lower than for placebo, and the difference was sustained. This result showed that the agent bacitracin zinc was surprisingly effective to reduce IOP in the glaucoma model.

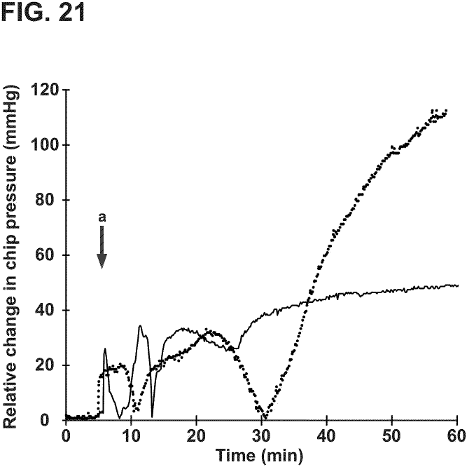

FIG. 22 shows that agent levetiracetam reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 22, the IOP for placebo (dashed line) increased greatly after injection of the placebo sample. The IOP rose steadily to a maximum pressure of about 55 mm Hg. To the contrary, the IOP after injection of the agent levetiracetam (solid line) was up to 62% lower than for placebo, and the difference was sustained. This result showed that the agent levetiracetam was surprisingly effective to reduce IOP in the glaucoma model.

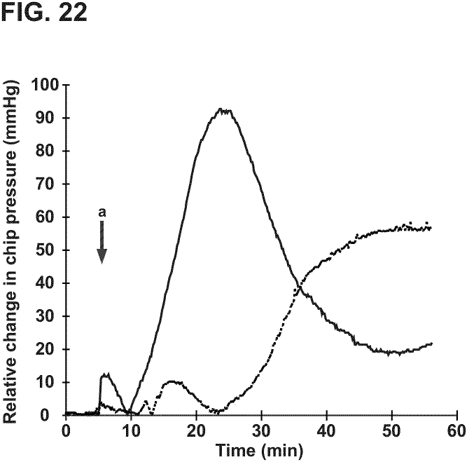

FIG. 23 shows that compound ombitasvir was a negative control for intraocular pressure (IOP) in a glaucoma model. The compound was tested by controlling flow and measuring relative IOP using in a device of this invention. The compound was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 23, the IOP for placebo (dashed line) increased greatly after injection of the placebo sample. The IOP rose steadily to a maximum pressure of about 110 mm Hg. However, the IOP after injection of ombitasvir (solid line) was significantly higher than for placebo. This result showed that ombitasvir was a negative control that did not reduce IOP in the glaucoma model.

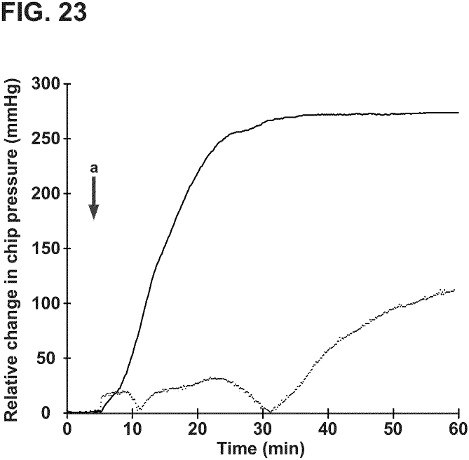

FIG. 24 shows that agent boceprevir reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 24, the IOP for placebo (dashed line) increased greatly after injection of the placebo sample. The IOP rose steadily to a maximum pressure of about 112 mm Hg. To the contrary, the IOP after injection of the agent boceprevir (solid line) was up to 67% lower than for placebo, and the difference was sustained. This result showed that the agent boceprevir was surprisingly effective to reduce IOP in the glaucoma model.

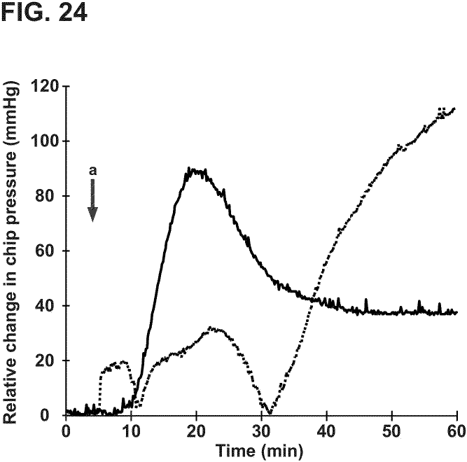

FIG. 25 shows that agent rapastinel TFA reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 25, the IOP for placebo (dashed line) increased greatly after injection of the placebo sample. The IOP rose steadily to a maximum pressure of about 57 mm Hg. To the contrary, the IOP after injection of the agent rapastinel TFA (solid line) was up to 82% lower than for placebo, and the difference was sustained. This result showed that the agent rapastinel TFA was surprisingly effective to reduce IOP in the glaucoma model.

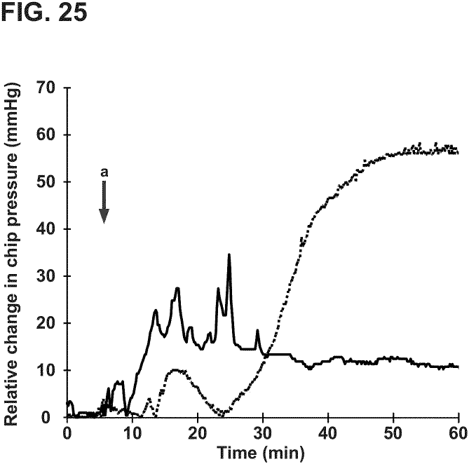

FIG. 26 shows that agent pramiracetam reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 26, the IOP for placebo (dashed line) increased greatly after injection of the placebo sample. The IOP rose steadily to a maximum pressure of about 57 mm Hg. To the contrary, the IOP after injection of the agent pramiracetam (solid line) was up to 44% lower than for placebo, and the difference was sustained. This result showed that the agent pramiracetam was surprisingly effective to reduce IOP in the glaucoma model.

FIG. 27 shows the dose-response behavior of the compound bivalirudin TFA on reducing intraocular pressure (IOP) in a bovine vitreous glaucoma model.

FIG. 28 shows a plan view of a microfluidic chip embodiment of this invention. In this format, a silicon wafer master 101 is printed with three microfluidic channel chip patterns 103. A silicon wafer 101 can be used as a substrate. Photoresist can be poured onto the substrate and exposed to UV light, which forms the pattern of the microfluidic chips 103. Together, the wafer and photoresist form a mold onto which PDMS can be poured. Once set, the PDMS can be peeled off the mold, giving three casts of microfluidic chips per wafer. These casts can be adhered to glass slides to form the final microfluidic chips.

FIG. 29 shows a plan view of a microfluidic chip insert in an embodiment of a device of this invention. The chip has two restriction channels 203, in this example each 2500 um wide and 25,000 um in length. The restriction channels 203 contain pillars of various diameters and spacing, shown by circles. The chip has a third uniform flow channel 205 having pillars of uniform size and spacing which do not significantly restrict the flow. The chip has an inlet reservoir 201 and an outlet reservoir 207, which also contain larger pillars. The dashed arrow shows the direction of flow from the inlet reservoir towards the outlet reservoir.

FIG. 30 shows a plan view corresponding to FIG. 29. FIG. 30 shows PDMS polymeric pillars 301 of various sizes represented by circles. The flow of biofluid through three channels is shown by dashed arrows.

FIG. 31 shows a plan view corresponding to the inlet reservoir of FIG. 29. FIG. 31 shows pillars 401 represented by circles. The flow of biofluid through three channels is shown by dashed arrows.

FIG. 32 shows a plan view corresponding to the inlet reservoir region of FIG. 29. FIG. 32 shows pillars 501 represented by circles. The flow of biofluid through three channels is shown by dashed arrows.

FIG. 33 shows a plan view corresponding to the channel region of FIG. 29. FIG. 33 shows pillars 601 represented by circles. The flow of biofluid through a channel is shown by a dashed arrow. The microfluidic channel device of this invention may have regions of different size and/or spacing of pillars or obstructions for creating turbulent or restricted flow.

FIG. 34 shows an expanded plan view corresponding to the channel region of FIG. 29. FIG. 34 shows pillars 701 represented by circles. The flow of biofluid through a channel is shown by a dashed arrow. This view shows a transition from 50 um gaps between pillars to 25 um gaps in a restriction channel.

FIG. 35 shows an expanded plan view corresponding to the channel region of FIG. 29. FIG. 35 shows pillars 801 represented by circles. The flow of biofluid through a channel is shown by a dashed arrow. This view shows a transition from larger to smaller gaps between pillars in a restriction channel.

FIG. 36 shows an expanded plan view corresponding to the channel region of FIG. 29. FIG. 36 shows pillars 901 represented by circles. The flow of biofluid through a channel is shown by a dashed arrow.

FIG. 37 shows an expanded plan view corresponding to the channel region of FIG. 29. FIG. 37 shows pillars 1001 represented by circles. The flow of biofluid through a channel is shown by a dashed arrow. This view shows channels having regions of blunt pillar obstructions 1001 which can create turbulent flow.

FIG. 38 shows an expanded plan view corresponding to the outlet reservoir 1107 of FIG. 29. FIG. 38 shows pillars 1101, 1103, and 1105 of various sizes. The flow of biofluid through a channel is shown by a dashed arrow. In this embodiment, the outer restriction channels each contain a barrier 1102 formed by very small and closely-spaced pillars.

FIG. 39 shows an expanded plan view corresponding to the inlet reservoir 1201 of FIG. 29. FIG. 39 shows pillars 1203 of various sizes. Outer restriction channel 1207 contains pillars of varying size and spacing. Uniform flow channel 1205 contains pillars of uniform size and spacing. The direction of flow of biofluid through an outer channel is shown by a dashed arrow.

FIG. 40 shows a plan view of a microfluidic chip in an embodiment of a device of this invention. Three microfluidic inserts are shown. The direction of flow of biofluid is shown by a dashed arrow.

FIG. 41 shows a perspective view of an embodiment of a microfluidic channel device of this invention having blunt pillar obstructions 1401 to flow. FIG. 41 is an expansion of FIG. 42. The direction of flow of biofluid is shown by dashed arrows.

FIG. 42 shows a perspective view of an embodiment of a microfluidic channel device of this invention. FIG. 42 shows a view corresponding to the channel region of FIG. 29. FIG. 42 shows blunt pillar obstructions 1501 of varying spacing in a restriction channel. In this embodiment, a restriction channel can have pillar obstructions 1501 organized in bands of varying spacing between the pillars. The direction of flow of biofluid is shown by a dashed arrow.

FIG. 43 shows an elevation side view of a microfluidic chip embodiment of this invention. The inlet reservoir 1605 is in fluid communication with a fluid line 1601 for introducing biofluid and/or other fluid into the reservoir. The fluid line 1601 passes through a probe 1602, probe adapter 1603, and hole 1604 defined in a glass cover slide. The biofluid passes through the inlet reservoir 1605 to reach the microfluidic channel 1606. The direction of flow of biofluid is shown by a dashed arrow.

FIG. 44 shows an expanded plan view corresponding to the inlet region of FIG. 29, and the position of a probe 1602 of FIG. 43. The direction of flow of biofluid is shown by a dashed arrow.

FIG. 45 shows an elevation side view of a microfluidic chip 1614 embodiment of this invention. The inlet reservoir is in fluid communication with a fluid line 1601 for introducing biofluid into the reservoir. The fluid line 1601 passes through a probe 1602, probe adapter 1603, and hole 1604 defined in a glass cover slide 1613. The biofluid passes through the inlet reservoir to reach the microfluidic channel 1606 and flow to the outlet reservoir 1607. A probe adjuster 1612 can be provided to adjust the height of the probe 1602 to create a good seal with the probe adapter 1603 and hole 1604. The direction of flow of biofluid is shown by a dashed arrow.

FIG. 46 shows an expanded plan view corresponding to the channel region of FIG. 29. FIG. 46 shows pillars 1701 represented by circles. For this embodiment, some representative lengths of regions of pillar bands in the outer channel are shown in micrometers.

FIG. 47 shows a micrograph of an expanded plan view corresponding to the channel region of FIG. 29. FIG. 47 shows pillars as dots. For this embodiment, some representative lengths of regions of pillar bands in the outer channel are shown in micrometers. The direction of flow of biofluid is shown by a dashed arrow.

FIG. 48 shows a plan view of an embodiment of a microfluidic device corresponding to FIG. 29. Biofluid can be introduced with a delivery probe 2201 to the inlet region reservoir 2202. The direction of flow of biofluid to the outlet reservoir region 2203 is shown by a dashed arrow. An expansion view for this embodiment shows some representative lengths of regions of pillar bands in the outer channel in micrometers. For this embodiment, dotted lines in the expansion view show possible tortuous paths of biofluid amongst the obstructions.

FIG. 49 shows an embodiment of a microfluidic system of this invention having a processor, a fluid drive unit, a fluid source unit, a sensor unit, an on-chip unit, and an off-chip unit.

### DETAILED DESCRIPTION OF THE INVENTION

This invention provides methods and compositions for treating glaucoma diseases. In some aspects, a range of compounds for treating glaucoma diseases are provided.

In further aspects, this invention provides methods and compositions for reducing intraocular pressure and increasing ocular outflows in glaucoma subjects. Aspects of this invention can reduce formation and presence of extracellular features and structures in ocular humor.

In additional aspects, this disclosure provides therapeutic compositions for glaucoma. The therapeutic compositions can reduce intraocular pressure in glaucoma, therefore surprisingly reducing risk of vision loss in glaucoma.

Embodiments of this invention further provide devices for measuring and characterizing glaucoma extracellular features, as well as intraocular pressure and ocular outflows.

In certain embodiments, this invention can provide compositions and methods for therapeutics and treatment of primary open-angle glaucoma (POAG), as well as testing of POAG aqueous humor specimens.

In further embodiments, glaucoma-associated extracellular matrix bodies (EMB) can be detected and measured. In additional embodiments, glaucoma-associated EMB may be reduced by compounds and compositions of this disclosure. In certain embodiments, glaucoma-associated EMB can contain glaucoma-associated-EV-complexes.

Extracellular matrix bodies or complexes of this disclosure may be composed of various biomolecules or complexed particles, and may have diameters ranging from about 0.5 to about 5,000, or from 0.5 to 1,000, or from 1 to 200, or from 1 to 100, or from 1 to 50, or from 1 to 25, or from 1 to 10, or from 1 to 5 micrometers.

In additional embodiments, compositions and methods of this invention can be used in therapies to reduce intraocular pressure (IOP) and/or increase ocular outflows.

Embodiments of this invention further contemplate methods for treating glaucoma diseases.

In certain aspects, a glaucoma disease may be treated by administering an agent active for ameliorating, alleviating, inhibiting, lessening, delaying, and/or preventing at least one symptom or condition of a glaucoma disorder.

### Glaucoma

Without wishing to be bound by theory, abnormal regulation of aqueous flow through the trabecular meshwork of the eye may be associated with elevated IOP. The extracellular matrix of the trabecular meshwork (TM) can be a barrier that may isolate the ocular fluid outflow. Ultrastructural and/or extracellular features or bodies in the aqueous humor of patients with glaucoma that are physically larger than the fenestrations of the juxtacanalicular (JCT) outlet, or that of other TM tissues, can block the TM. Ultrastructural and/or extracellular features or bodies in the aqueous humor can include structures based on extracellular matrix bodies (EMB). In some aspects, extracellular matrix bodies may contain various particles, biomolecules, or vesicles.

Some modalities for glaucoma are given in PCT/US2019/052310, which is hereby incorporated by reference in its entirety for all purposes.

Glaucoma disorders, referred to herein as “glaucoma,” that can be treated with the methods and compositions disclosed herein include preglaucoma open angle with borderline findings, open angle, low risk, glaucoma suspect, anatomical narrow angle primary angle closure suspect, steroid responder, ocular hypertension, primary angle closure without glaucoma damage (PAS or high IOP with no optic nerve or visual field loss), unspecified open-angle glaucoma, primary open-angle glaucoma, chronic simple glaucoma, low-tension glaucoma, pigmentary glaucoma, capsular glaucoma with pseudo-exfoliation of lens, residual stage of open-angle glaucoma, unspecified primary angle-closure glaucoma, acute angle-closure glaucoma attack, chronic angle-closure glaucoma, intermittent angle-closure glaucoma, residual stage of angle-closure glaucoma, glaucoma secondary to eye trauma, glaucoma secondary to eye inflammation, glaucoma secondary to other eye disorders including, retinal vascular occlusions, diabetes type 1 complicated, diabetes type 2 complicated, disorders of lens, disorders of intraocular lens, disorders after other ocular symptoms, neoplasms, benign neoplasms, or malignant. Also included is glaucoma secondary to drugs, glaucoma with increased episcleral venous pressure, hypersecretion glaucoma, aqueous misdirection malignant glaucoma, glaucoma in diseases classified elsewhere, congenital glaucoma, Axenfeld’s anomaly, buphthalmos, glaucoma of childhood, glaucoma of newborn, hydrophthalmos, keratoglobus, congenital glaucoma macrocornea with glaucoma, macrophthalmos in congenital glaucoma, megalocornea with glaucoma, absolute glaucoma. Also included are adverse effect of ophthalmological drugs and preparations, acute follicular conjunctivitis, adverse effect of carbonic anhydrase inhibitors, and adverse effect of under dosing of ophthalmological drugs and preparations.

Glaucoma disorders include preglaucoma open angle with borderline findings, open angle, low risk, anatomical narrow angle primary angle closure suspect, steroid responder, ocular hypertension, primary angle closure without glaucoma damage (pas or high iop with no optic nerve or visual field loss), unspecified open-angle glaucoma, primary open-angle glaucoma chronic simple glaucoma, low-tension glaucoma, pigmentary glaucoma, capsular glaucoma with pseudo-exfoliation of lens, residual stage of open-angle glaucoma, unspecified primary angle-closure glaucoma, acute angle-closure glaucoma attack, chronic angle-closure glaucoma, intermittent angle-closure glaucoma, residual stage of angle-closure glaucoma, glaucoma secondary to eye trauma, glaucoma secondary to eye inflammation glaucoma secondary to other eye disorders including; retinal vascular occlusions, diabetes type 1 complicated, diabetes type 2 complicated, disorders of lens, disorders of intraocular lens, disorders after other ocular symptoms, neoplasms, benign neoplasms, or malignant. glaucoma secondary to drugs, glaucoma with increased episcleral venous pressure, hypersecretion glaucoma, aqueous misdirection malignant glaucoma, glaucoma in diseases classified elsewhere. congenital glaucoma; axenfeld’s anomaly, buphthalmos, glaucoma of childhood, glaucoma of newborn, hydrophthalmos, keratoglobus, congenital, with glaucoma macrocornea with glaucoma macrophthalmos in congenital glaucoma megalocornea with glaucoma. absolute glaucoma, adverse effect of ophthalmological drugs and preparations, acute follicular conjunctivitis, adverse effect of carbonic anhydrase inhibitors, adverse effect of under dosing of ophthalmological drugs and preparations.

In some embodiments, a composition of this disclosure can be administered extraocularly, or by ocular implants. Systemic administration can also be achieved via topical eye drops.

Administering may also be carried out to deliver the therapeutic agent to the subject’s ocular cells or tissue, which may be topical administration, systemic administration, periocular administration, or intraocular administration. Intraocular administration may be carried out via intracameral administration, intravitreal administration, or subretinal administration.

In certain embodiments, a composition of this disclosure can be administered intraocularly. Systemic administration can also be achieved via intracameral administration, intravitreal administration, or subretinal administration.

In some embodiments, a composition of this disclosure can be administered systemically. Systemic administration can be achieved via intravenous administration, oral administration, intraarterial administration, inhalation, intranasal administration, intraperitoneal administration, intra-abdominal administration, subcutaneous administration, intra-articular administration, intrathecal administration, transdural administration, transdermal administration, submucosal administration, sublingual administration, enteral administration, parenteral administration, percutaneous administration, periarticular administration, or intraventricular administration.

### Active Agents

In some embodiments, active agents for use in treating glaucoma include nucleoside phosphates and/or nucleoside phosphonates. Some examples of nucleoside phosphonates are given in WO2007130783, including Table 1 therein, hereby incorporated by reference. Some example of nucleoside phosphonate esters are given in US 8,835,630 and US2014/0364397, hereby incorporated by reference.

In some aspects, nucleoside phosphates and/or nucleoside phosphonates for use as active agents in treating glaucoma by local administration to ocular tissue are not subject to metabolic oxidation or degradation. Further, nucleoside phosphates and/or nucleoside phosphonates for use as active agents in treating glaucoma by local administration to ocular tissue avoids any known systemic or thoracic organ related toxicity. Thus, the nucleoside phosphates and/or nucleoside phosphonates of this disclosure for use as active agents in treating glaucoma are surprisingly active.

In some embodiments, active agents for use in treating glaucoma include compounds shown in Formula I.

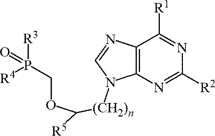

wherein
R1 is selected from H, Cl, amino, alkylamino, cycloalkylamino, cycloalkyl-alkylamino, aminoalkylamino, carboxyalkylamino, benzylamino, ═O;
R2 is selected from H, Cl, ═O, NR6R7, alkylamino, arylamino, benzylamino, 2-pyridinylamino, wherein R6, R7 are selected from H, alkyl, cycloalkyl, aryl;
R3,R4 are independently selected from OH, alkoxy, aminoalkoxy, hydroxyalkoxy, carboxyalkoxy, haloalkoxy, alkoxyalkoxy, carboxyalkylcarboxyl, carboxyalkenylcarboxyl, benzyloxy, amino, alkylamino, carboxyalkylamino, carboxylate-alkylamino, wherein R3,R4 may connect to form a loop;
R5 is selected from H, alkyl, hydroxyalkyl, cycloalkyl, aryl;
n is 1-5;
and pharmaceutically-acceptable prodrugs, esters and salts thereof.

In some embodiments, active agents for use in treating glaucoma include compounds shown in Formula I, wherein
R¹ is selected from H, Cl, amino, alkylamino, cycloalkylamino;
R² is selected from H, Cl, ═O;
R³, R⁴ are independently selected from OH, alkoxy, aminoalkoxy, hydroxyalkoxy, haloalkoxy, alkoxyalkoxy, amino, alkylamino, cycloalkylamino, wherein R³, R⁴ may connect to form a loop;
R⁵ is selected from H, alkyl, cycloalkyl, hydroxyalkyl, aryl;
n is 1-5;
and pharmaceutically-acceptable prodrugs, esters and salts thereof.

In some embodiments, an active agent for use in treating glaucoma can be adefovir, as shown in Formula II.

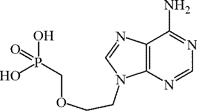 [SMILES: Nc1ncnc2c1ncn2CCOCP(=O)(O)O]

which is ((2-(6-amino-9H-purin-9-yl)ethoxy)methyl)phosphonic acid, also known as ((2-(6-Amino-9H-purin-9-yl)ethoxy)methyl)phosphonic acid, and 9-(2-Phosphonylmethoxyethyl)adenine. Adefovir may be used in a prodrug form such as adefovir dipivoxil. Adefovir may be used in a pharmaceutically-acceptable salt form.

In some embodiments, an active agent for use in treating glaucoma can be pradefovir Formula III, which may be used in a prodrug form or in a pharmaceutically-acceptable salt form.

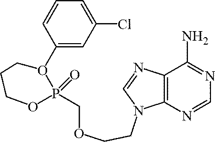

which is 2-((2-(6-amino-9H-purin-9-yl)ethoxy)methyl)-1-(3-chlorophenyl)-113,3,2-dioxaphosphinane 2-oxide.

In some embodiments, an active agent for use in treating glaucoma can be tenofovir Formula IV, which may be used in a prodrug form or in a pharmaceutically-acceptable salt form.

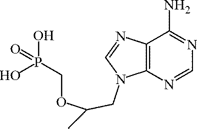 [SMILES: CC(Cn1cnc2c(N)ncnc21)OCP(=O)(O)O]

which is (((1-(6-amino-9H-purin-9-yl)propan-2-yl)oxy)methyl)phosphonic acid.

Examples of active agents for use in treating glaucoma include compounds shown in Formula V.

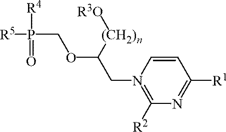

wherein
R¹ is selected from H, Cl, ═O, amino, alkylamino, cycloalkylamino, cycloalkyl-alkylamino, aminoalkylamino, carboxyalkylamino, benzylamino;
R² is selected from H, Cl, NR⁶R⁷, and alkylamino, cycloalkylamino, arylamino, benzylamino, 2-pyridinylamino, wherein R⁶, R⁷ are selected from H, alkyl, cycloalkyl, aryl;
R³ is selected from H, alkyl, cycloalkyl, aryl;
R⁴, R⁵ are independently selected from OH, alkoxy, aminoalkoxy, hydroxyalkoxy, carboxyalkoxy, haloalkoxy, alkoxyalkoxy, carboxylalkenylcarboxyl, benzyloxy, amino, alkylamino, cycloalkylamino, carboxylalkylamino, carboxylate-alkylamino, wherein R³, R⁴ may connect to form a loop;
n is 1-5;
and pharmaceutically-acceptable prodrugs, esters and salts thereof.

Examples of active agents for use in treating glaucoma include compounds shown in Formula V, wherein
R¹ is selected from H, Cl, ═O, amino, alkylamino, cycloalkylamino;
R² is selected from H, Cl, amino, alkylamino, cycloalkylamino;
R³ is selected from H, alkyl, cycloalkyl, aryl;
R⁴, R⁵ are independently selected from OH, alkoxy, aminoalkoxy, hydroxyalkoxy, alkoxyalkoxy, benzyloxy, amino, alkylamino;
n is 1-5;
and pharmaceutically-acceptable prodrugs, esters and salts thereof.

In some embodiments, an active agent for use in treating glaucoma can be cidofovir Formula VI, which may be used in a prodrug form, ester form, or in a pharmaceutically-acceptable salt form.

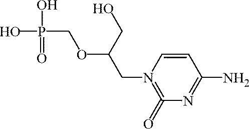

which is (((1-(4-amino-2-oxopyrimidin-1(2H)-yl)-3-hydroxypropan-2-yl)oxy)methyl)phosphonic acid.

In some embodiments, an active agent for use in treating glaucoma can be brincidofovir Formula VII, which may be used in a prodrug form, ester, or in a pharmaceutically-acceptable salt form.

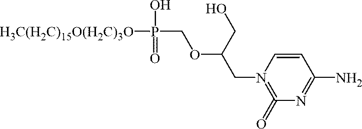

which is 3-(hexadecyloxy)propyl hydrogen (((1-(4-amino-2-oxopyrimidin-1(2H)-yl)-3-hydroxypropan-2-yl)oxy)methyl)phosphonate.

In some embodiments, active agents for use in treating glaucoma include compounds shown in Formula VIII.

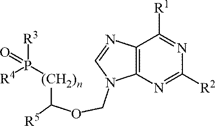

wherein
R¹ is selected from H, Cl, ═O, amino, alkylamino, cycloalkylamino, cycloalkyl-alkylamino, aminoalkylamino, carboxyalkylamino, benzylamino;
R² is selected from H, Cl, NR⁶R⁷, alkylamino, cycloalkylamino, arylamino, benzylamino, 2-pyridinylamino, wherein R⁶, R⁷ are selected from H, alkyl, cycloalkyl, aryl;
R³, R⁴ are independently selected from OH, alkoxy, aminoalkoxy, hydroxyalkoxy, carboxylalkoxy, haloalkoxy, alkoxyalkoxy, carboxylalkylcarboxyl, carboxylalkenylcarboxyl, benzyloxy, amino, alkylamino, carboxylalkylamino, carboxylate-alkylamino, wherein R³,R⁴ may connect to form a loop;
R⁵ is selected from H, alkyl, hydroxyalkyl, aminoalkyl, cycloalkyl, aryl;
n is 1-5;
and pharmaceutically-acceptable prodrugs, esters and salts thereof;
preferably excluding acyclovir for use in treating glaucoma;
more preferably excluding acyclovir and all pharmaceutically acceptable prodrugs, esters and salts thereof for use in treating glaucoma; even more preferably excluding acyclovir and all pharmaceutically acceptable prodrugs, esters and salts thereof for any use.

In some embodiments, active agents for use in treating glaucoma include compounds shown in Formula VIII, wherein
R¹ is selected from H, Cl, =O, amino, alkylamino, cycloalkylamino;
R² is selected from H, Cl, NR⁶R⁷, alkylamino, cycloalkylamino, wherein R⁶, R⁷ are selected from H, alkyl, cycloalkyl, aryl;
R³, R⁴ are independently selected from OH, alkoxy, aminoalkoxy, hydroxyalkoxy, haloalkoxy, alkoxyalkoxy, benzyloxy, amino, alkylamino, wherein R³, R⁴ may connect to form a loop;
R⁵ is selected from H, alkyl, cycloalkyl, hydroxyalkyl, aryl;
n is 1-5;
and pharmaceutically-acceptable prodrugs, esters and salts thereof;
preferably excluding acyclovir for use in treating glaucoma;
more preferably excluding acyclovir and all pharmaceutically acceptable prodrugs, esters and salts thereof for use in treating glaucoma; even more preferably excluding acyclovir and all pharmaceutically acceptable prodrugs, esters and salts thereof for any use.

In some embodiments, an active agent for use in treating glaucoma can be acyclovir Formula IX, which may be used in a prodrug form, ester or in a pharmaceutically-acceptable salt form.

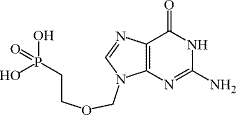 [SMILES: Nc1nc2c(ncn2COCCP(=O)(O)O)c(=O)[nH]1]

which is (2-((2-amino-6-oxo-1,6-dihydro-9H-purin-9-yl)methoxy)ethyl)phosphonic acid.

In some embodiments, active agents for use in treating glaucoma include acediasulfone, aceturate, acetyl sulfametossipirazine, acetyl sulfamethoxypyrazine, acranil, acyclovir, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, bacitracin, benzoylpas, benzyl penicillin acid, benzyl sulfamide, bicozamycin, bipenam, brodimoprim, capreomycin, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chibrorifamycin, chloramphenicol, chlorotetracycline, cidofovir, cinoxacin, ciprofloxacin, claritromycin, clavulanic acid, clinafloxacin, clindamycin, clofazimine, clofoctal, clometocillin, clomocycline, cloxacillin, cloxyquin, colistin, cyclacilline, cycloserine, cytarabine, danoflaxcin, dapsone, deoxycycline, deoxydihydrostreptomycin, dibekacin, dicloxacillin, didanosine, dideoxyadenosine, difloxacin, dihydrostreptomycin, dimetridazole, diminazene, dirirtomycin, duramycin, edoxudine, eflornithine, enrofloxacin, enviomycin, epicillin, erythromycin, etacillin, ethambutol, ethionamide, famcyclovir, fenbecillin, fleroxacin, flomoxef, floxacillin, floxuridine, flumequine, n-formamidoylthienamycin, furonazide, fortimycin, furazolium chloride, ganciclovir, gentamycin, glyconiazide, gramicidin, grepafloxacin, guamecycline, halofuginone, hetacillin, homidium, hydroxyl-stilbamidine, ibostamycin, idoxuridine, imidocarb, imipenam, indinavir, ipronidazole, isoniazide, josamycin, kanamycin, kethoxal, lamivudine, lauroguadine, lenampicillin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl) sulfanilanilide, mezlocillin, meziocillin, micronomycin, midecamycin A.sub.1, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin 0, penicillin V, penciclovir, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, podophyllotoxin, polymyxin B, profiromycin, propamidine, propicillin, protionamide, puraltadone, puromycin, pyrazinamide, pyrimethamine, quinacillin, quinacrine, quinapyramine, quintine, ribostamycin, rifabutine, ribavirine, rifamide, rifampin, rifamycin, rifanpin, rifapentine, rifaxymine, rimantadine, ritipenem, rokitamycin, rolitetracycline, rosamycin, rufloxacin, salazosulfadimidine, salinazid, sancycline, saquinavir, sarafloxacin, sedacamycin, secnidazole, sisomycin, sorivudine, sparfloxacin, spectinomycin, spiramycin, spiramycin I, spiramycin II, spiramycin III, stavudine, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N.sup.4-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, tinidazole, tobramycin, tosufloxacin, trifluridine, trimethoprim, troleandromycin, trospectomycin, trovafloxacin, tubercidine, miokamycin, oleandomycin, troleandromycin, vancomycin, valacyclovir, vidarabine, verazide, viomycin, virginiamycin, zalcitabine, zidovudine, and combinations and pharmaceutically-acceptable salts thereof.

In some embodiments, active agents for use in treating glaucoma include aminoglycosides, fluoroquinolones, tetralides, cephalosporins, and combinations and pharmaceutically-acceptable salts thereof.

In some embodiments, active agents for use in treating glaucoma include tobramycin, gentamicin, ciprofloxacin, norfloxacin, ofloxacin, sparfloxacin, and combinations and pharmaceutically-acceptable salts thereof.

In some embodiments, active agents for use in treating glaucoma include aminoglycosides including amikacin, apramycin, arbekacin, bambermycins, butirosin, dibekacin, dihydrostreptomycin, fortimicin, gentamicin, isepamicin, kanamycin, micronomicin, neomycin, neomycin undecylenate, netilmicin, paromomycin, ribostamycin, sisomicin, spectinomycin, streptomycin, tobramycin, trospectomycin, and combinations and pharmaceutically-acceptable salts thereof.

In some embodiments, active agents for use in treating glaucoma include amphenicols including azidamfenicol, chloramphenicol, florfenicol, thiamphenicol, and combinations and pharmaceutically-acceptable salts thereof.

In some embodiments, active agents for use in treating glaucoma include ansamycins including rifamide, rifampin, rifamycin, rifapentine, rifaximin, and combinations and pharmaceutically-acceptable salts thereof.

In some embodiments, active agents for use in treating glaucoma include β-lactams including carbacephems such as loracarbef, carbapenems such as biapenem, imipenem, meropenem, and panipenem, and combinations and pharmaceutically-acceptable salts thereof.

In some embodiments, active agents for use in treating glaucoma include cephalosporins such as cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, and pivcefalexin, and combinations and pharmaceutically-acceptable salts thereof.

In some embodiments, active agents for use in treating glaucoma include cephamycins such as cefbuperazone, cefmetazole, cefininox, cefotetan, and cefoxitin, and combinations and pharmaceutically-acceptable salts thereof.

In some embodiments, active agents for use in treating glaucoma include monobactams such as aztreonam, carumonam, tigemonam, oxacephems, flomoxef, and moxalactam, and combinations and pharmaceutically-acceptable salts thereof.

In some embodiments, active agents for use in treating glaucoma include penicillins such as amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, and ticarcillin, and combinations and pharmaceutically-acceptable salts thereof.

In some embodiments, active agents for use in treating glaucoma include macrolides such as azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, and troleandomycin, and combinations and pharmaceutically-acceptable salts thereof.

In some embodiments, active agents for use in treating glaucoma include polypeptides such as amphomycin, bacitracin, capreomycin, colistin, enduracidin, enviomycin, fusafungine, gramicidins, gramicidin, mikamycin, polymyxins, pristinamycin, ristocetin, teicoplanin, thiostrepton, tuberactinomycin, tyrocidine, tyrothricin, vancomycin, viomycin, virginiamycin, and zinc bacitracin, and combinations and pharmaceutically-acceptable salts thereof.

In some embodiments, active agents for use in treating glaucoma include tetracyclines such as apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, and tetracycline, and combinations and pharmaceutically-acceptable salts thereof.

In some embodiments, active agents for use in treating glaucoma include 2,4-diaminopyrimidines such as brodimoprim, tetroxoprim, trimethoprim, and combinations and pharmaceutically-acceptable salts thereof.

In some embodiments, active agents for use in treating glaucoma include nitrofurans such as furaltadone, furazolium chloride, nifuradene, nifuratel, nifurfoline, nifurpirinol, nifurprazine, nifurtoinol, nitrofurantoin, and combinations and pharmaceutically-acceptable salts thereof.

In some embodiments, active agents for use in treating glaucoma include quinolones such as cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin, and combinations and pharmaceutically-acceptable salts thereof.

In some embodiments, active agents for use in treating glaucoma include sulfonamides such as acetyl sulfamethoxypyrazine, benzylsulfamide, chloramine-b, chloramine-t, dichloramine t, n2-formylsulfisomidine, n4- β -d-glucosylsulfanilamide, mafenide, 4′ -(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, n4-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole, and combinations and pharmaceutically-acceptable salts thereof.

In some embodiments, active agents for use in treating glaucoma include sulfones such as acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone, and combinations and pharmaceutically-acceptable salts thereof.

In some embodiments, active agents for use in treating glaucoma include clofoctol, hexedine, methenamine, methenamine anhydromethylene-citrate, methenamine hippurate, methenamine mandelate, methenamine sulfosalicylate, nitroxoline, taurolidine, xibornol, and combinations and pharmaceutically-acceptable salts thereof.

In some embodiments, active agents for use in treating glaucoma include compounds shown in Formula X.

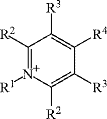

wherein
R¹ is selected from alkyl, cycloalkyl, aminoalkyl, acylalkyl, benzyl, alkenyl, alkynyl, wherein R¹ is terminated with H, a carbon-carbon double bond, or a methacryloyloxy group;
R², R³ are independently selected from H, halo, alkyl;
R⁴ is selected from H, OH, alkoxy, alkylalkoxy, aminoalkoxy, hydroxyalkoxy, carboxyalkoxy, haloalkoxy, alkoxyalkoxy, benzyl, amino, alkylamino, carboxyalkylamino, carboxylate-alkylamino;
and pharmaceutically-acceptable prodrugs, esters and salts thereof;
preferably excluding cetylpyridinium for use in treating glaucoma;
more preferably excluding cetylpyridinium and all pharmaceutically acceptable prodrugs, esters and salts thereof for use in treating glaucoma; even more preferably excluding cetylpyridinium and all pharmaceutically acceptable prodrugs, esters and salts thereof for any use.

In some embodiments, active agents for use in treating glaucoma include compounds shown in Formula X; wherein
R¹ is selected from alkyl, cycloalkyl, aminoalkyl, alkenyl, alkynyl, wherein R¹ is terminated with H, a carbon-carbon double bond, or a methacryloyloxy group;
R², R³ are independently selected from H, halo, alkyl;
R⁴ is selected from H, OH, alkoxy, amino, alkylamino, cycloalkylamino;
and pharmaceutically-acceptable prodrugs, esters and salts thereof;
preferably excluding cetylpyridinium for use in treating glaucoma;
more preferably excluding cetylpyridinium and all pharmaceutically acceptable prodrugs, esters and salts thereof for use in treating glaucoma;
even more preferably excluding cetylpyridinium and all pharmaceutically acceptable prodrugs, esters and salts thereof for any use.

In some embodiments, active agents for use in treating glaucoma include compounds shown in Formula X wherein
R¹ is C(14-24)alkyl, C(14-24)alkenyl;
R², R³ are independently selected from H, halo, alkyl;
R⁴ is selected from H, OH, alkoxy, amino, alkylamino, cycloalkylamino;
and pharmaceutically-acceptable prodrugs, esters and salts thereof;
preferably excluding cetylpyridinium for use in treating glaucoma;
more preferably excluding cetylpyridinium and all pharmaceutically acceptable prodrugs, esters and salts thereof for use in treating glaucoma;
even more preferably excluding cetylpyridinium and all pharmaceutically acceptable prodrugs, esters and salts thereof for any use.

In some embodiments, active agents for use in treating glaucoma include compounds shown in Formula X wherein
R¹ is C(14-24)alkenyl;
R², R³ are independently selected from H, halo, alkyl;
R⁴ is selected from H, OH, alkoxy, amino, alkylamino, cycloalkylamino;
and pharmaceutically-acceptable prodrugs, esters and salts thereof.

In some embodiments, an active agent for use in treating glaucoma can be cetylpyridinium Formula XI, which may be used in a prodrug form, ester or in a pharmaceutically-acceptable salt form.

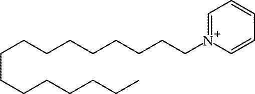 [SMILES: CCCCCCCCCCCCCCCC[n+]1ccccc1]

which is 1-hexadecylpyridin-1-ium.

In some embodiments, an active agent for use in treating glaucoma can be cetylpyridinium Formula XII, which may be used in a prodrug form, ester or in a pharmaceutically-acceptable salt form.

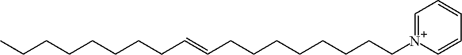 [SMILES: CCCCCCCC/C=C/CCCCCCCC[n+]1ccccc1]

which is C(18:1(9))alkenyl-pyridin-1-ium.

In some embodiments, an active agent for use in treating glaucoma can be cetylpyridinium Formula XIII, which may be used in a prodrug form, ester or in a pharmaceutically-acceptable salt form.

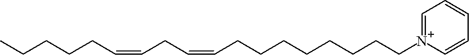 [SMILES: CCCCC/C=C\C/C=C\CCCCCCCC[n+]1ccccc1]

In some embodiments, an active agent for use in treating glaucoma can be cetylpyridinium Formula XIV, which may be used in a prodrug form, ester or in a pharmaceutically-acceptable salt form.

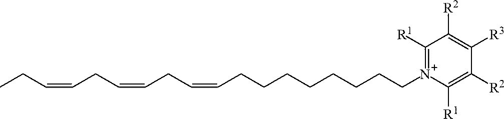

In some embodiments, active agents for use in treating glaucoma include cyclic polypeptides and pharmaceutically-acceptable prodrugs, esters and salts thereof.

In some aspects, cyclic polypeptides for use as active agents in treating glaucoma by local administration to ocular tissue are not subject to metabolic oxidation or degradation. Further, cyclic polypeptides for use as active agents in treating glaucoma by local administration to ocular tissue avoids any known systemic or thoracic organ related toxicity. Thus, the cyclic polypeptides of this disclosure for use as active agents in treating glaucoma are surprisingly active.

A cyclic polypeptide of this disclosure can be monocyclic, bicyclic, or may contain peptidic branches from a cyclic portion.

A cyclic polypeptide of this disclosure may have a cationic peptide ring.

In some embodiments, a cyclic polypeptide for use as active agent in treating glaucoma may have from 7-30 monomers, wherein the monomers comprise naturally-occurring or synthetic amino acid monomers. A cyclic polypeptide of this disclosure may have one or more cyclic portions, one or more monomer chains which are a side branch of a cyclic portion, and one or more lipophilic, amphiphilic, or amphoteric substituents.

A cyclic polypeptide of this disclosure can be a cyclic hepapeptide with a tripeptide side branch. The tripeptide side branch may be acylated at the N-terminus with an alkanoyl or alkenoyl chain.

Some cyclic polypeptides of this disclosure may be isolated from B. polmyxa.

Examples of a cyclic polypeptide of this disclosure include polymyxins A, B₁, B₂, C, D, E, and P. Polymyxin E is also known as colistin.

Examples of a cyclic polypeptide of this disclosure include polymyxin B sulphate salt and colistin methanesulphonate sodium salt. Colistin methanesulphonate sodium salt may be a prodrug form of colistin.

A cyclic polypeptide of this disclosure can be synthetic. Some compounds and methods for synthesis are given in Tang, J. Antibiotics, 2020, Vol. 73, pp. 158-166; Gallardo-Godoy, Molecules, 2019, Vol. 24, pp. 553-566; Kim, J. Microbiol. Biotechnol., 2015, Vol. 25, pp. 1015-1025.

Polymyxin B can be a mixture of at four or more components B1 to B4.

In some embodiments, active agents for use in treating glaucoma include compounds shown in Formula XV.

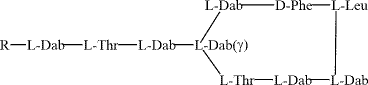

wherein R is selected from alkyl, cycloalkyl, aminoalkyl, alkenyl, alkynyl, alkanoyl, alkenoyl, wherein Dab is a diaminobutanoic acid monomer. In some embodiments, R is 6-methyloctanoyl (B₁), 6-methylheptanoyl (B₂), octanoyl (B₃), heptanoyl (B₄);

and pharmaceutically-acceptable prodrugs, esters and salts thereof.

In some embodiments, active agents for use in treating glaucoma include compounds shown in Formula XV;
wherein R is selected from alkyl, cycloalkyl, aminoalkyl, alkenyl, alkynyl, alkanoyl, alkenoyl. In some embodiments, R is 6-methyloctanoyl (B₁), 6-methylheptanoyl (B₂), octanoyl (B₃), heptanoyl (B₄);
and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

In some embodiments, active agents for use in treating glaucoma include compounds shown in Formula XVI.

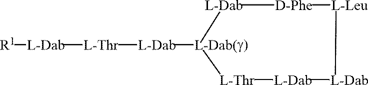

wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy;

and pharmaceutically-acceptable prodrugs, esters and salts thereof.

In some embodiments, active agents for use in treating glaucoma include compounds shown in Formula XVI;
wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy;
and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

In some embodiments, active agents for use in treating glaucoma include compounds shown in Formula XVI;

wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVI;
wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

In some embodiments, active agents for use in treating glaucoma include compounds shown in Formula XVI, wherein R¹ is a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVII.

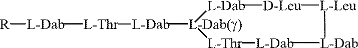

wherein R is selected from alkyl, cycloalkyl, aminoalkyl, alkenyl, alkynyl, alkanoyl, alkenoyl, and pharmaceutically-acceptable prodrugs, esters and salts thereof.

In some embodiments, active agents for use in treating glaucoma include compounds shown in Formula XVII;

wherein R is selected from alkyl, cycloalkyl, aminoalkyl, alkenyl, alkynyl, alkanoyl, alkenoyl, and pharmaceutically-acceptable prodrugs, esters and salts thereof.

In some embodiments, active agents for use in treating glaucoma include compounds shown in Formula XVIII.

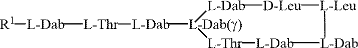

wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVIII;

wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVIII;

wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVIII;

wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVIII;

wherein R¹ is a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XVIII;

wherein R¹ is a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

In some embodiments, active agents for use in treating glaucoma include compounds shown in Formula XIX.

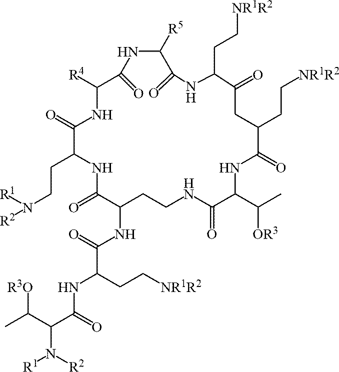

wherein
R¹, R² are independently selected from H, alkyl, aminoalkyl, hydroxyalkyl, carboxylalkyl;
R³ is selected from H, alkyl, aminoalkyl, cycloalkyl, hydroxyalkyl, carboxylalkyl, aryl;
R⁴ is selected from H, alkyl, aminoalkyl, cycloalkyl, hydroxyalkyl, carboxylalkyl, benzyl, aryl, aralkyl, cycloalkyl-alkyl;
R⁵ is selected from H, alkyl, aminoalkyl, cycloalkyl, hydroxyalkyl, carboxylalkyl, aryl;
and pharmaceutically-acceptable prodrugs, esters and salts thereof.

In some embodiments, active agents for use in treating glaucoma include compounds shown in Formula XIX, wherein
R¹, R² are independently selected from H, alkyl, aminoalkyl, cycloalkyl, hydroxyalkyl, carboxylalkyl, aryl;
R³ is selected from H, alkyl, aminoalkyl, cycloalkyl, hydroxyalkyl, carboxylalkyl, aryl;
R⁴ is selected from H, alkyl, aminoalkyl, cycloalkyl, hydroxyalkyl, carboxylalkyl, benzyl, aryl, aralkyl, cycloalkyl-alkyl;
R⁵ is selected from H, alkyl, aminoalkyl, cycloalkyl, hydroxyalkyl, carboxylalkyl, aryl; and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

In some embodiments, active agents for use in treating glaucoma include compounds shown in Formula XX.

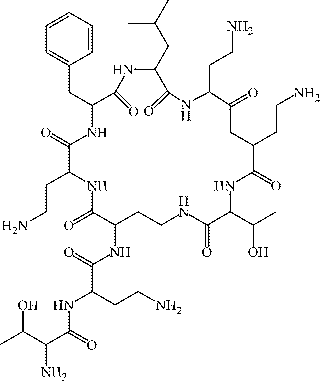 [SMILES: CC(C)CC1NC(=O)C(Cc2ccccc2)NC(=O)C(CCN)NC(=O)C(NC(=O)C(CCN)NC(=O)C(N)C(C)O)CCNC(=O)C(C(C)O)NC(=O)C(CCN)CC(=O)C(CCN)NC1=O]

and pharmaceutically-acceptable prodrugs, esters and salts thereof.

In some embodiments, active agents for use in treating glaucoma include compounds shown in Formula XX;
and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

In some embodiments, an active agent for use in treating glaucoma can be polymyxin B shown in Formula XXI, which may be used in a prodrug form or in a pharmaceutically-acceptable salt form.

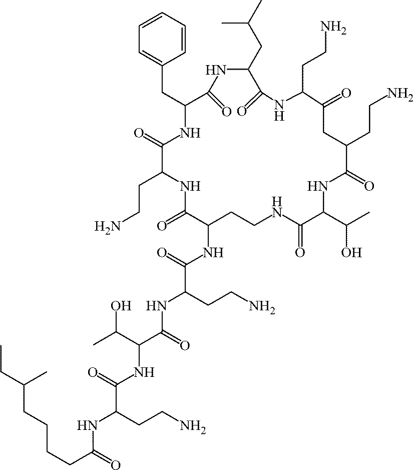 [SMILES: CCC(C)CCCCC(=O)NC(CCN)C(=O)NC(C(=O)NC(CCN)C(=O)NC1CCNC(=O)C(C(C)O)NC(=O)C(CCN)CC(=O)C(CCN)NC(=O)C(CC(C)C)NC(=O)C(Cc2ccccc2)NC(=O)C(CCN)NC1=O)C(C)O]

A cyclic polypeptide of this disclosure can be a cyclic hepapeptide with a tripeptide side branch. The tripeptide side branch may be acylated at the N-terminus with a 1-amino-2-methylbutyl-4,5-dihydro-1,3-thiazole-4-carboxyl, an alkanoyl, or an alkenoyl.

Some cyclic polypeptides of this disclosure may be isolated from B. subtilis var Tracy.

Examples of a cyclic polypeptide of this disclosure include bacitracins, and bacitracin A.

In some embodiments, active agents for use in treating glaucoma include compounds shown in Formula XXI.

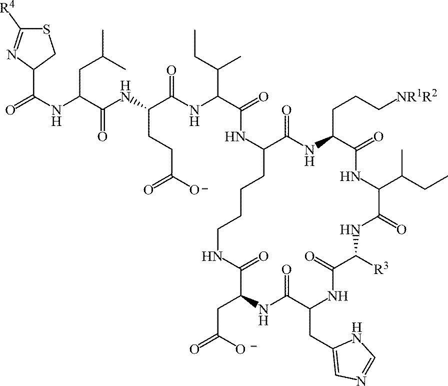

and pharmaceutically-acceptable prodrugs, esters and salts thereof; wherein
R¹, R² are independently selected from H, alkyl, aminoalkyl, cycloalkyl, hydroxyalkyl, carboxylalkyl, aryl;
R³ is selected from H, alkyl, cycloalkyl, aryl, benzyl, arylalkyl;
R⁴ is selected from H, alkyl, aminoalkyl, cycloalkyl, arylalkyl, aryl.

In some embodiments, active agents for use in treating glaucoma include bacitracin A shown in Formula XXII.

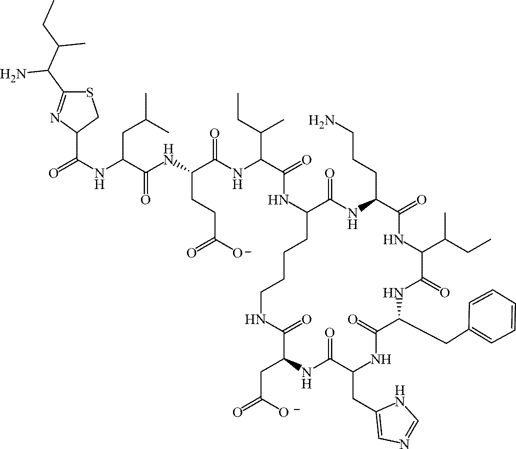 [SMILES: CCC(C)C(N)C1=NC(C(=O)NC(CC(C)C)C(=O)N[C@@H](CCC(=O)[O-])C(=O)NC(C(=O)NC2CCCCNC(=O)[C@H](CC(=O)[O-])NC(=O)C(Cc3cnc[nH]3)NC(=O)[C@@H](Cc3ccccc3)NC(=O)C(C(C)CC)NC(=O)[C@H](CCCN)NC2=O)C(C)CC)CS1]

and pharmaceutically-acceptable prodrugs, esters and salts thereof.

Embodiments of this invention further contemplate active agents for use in treating glaucoma having a range of structures based on natural or synthetic polypeptides.

In some embodiments, active agents for use in treating glaucoma include peptides being at least 75%, or 80%, or 85%, or 90%, or 95% identical to a reference polypeptide.

An active agent may further have conservative replacement of 1-5 peptidic monomers.

In some embodiments, a reference polypeptide can be bivalirudin, hirudin, or rapastinel.

In some embodiments, active agents for use in treating glaucoma include peptides being at least 75%, or 80%, or 85%, or 90%, or 95% identical to a reference polypeptide, where the active agent polypeptide may vary from a reference polypeptide by having 1-5 monomers selected from Lys, His, Arg, flanking one or both termini of the reference polypeptide.

In some embodiments, active agents for use in treating glaucoma include bivalirudin shown in Formula XXIII, and pharmaceutically-acceptable prodrugs, esters and salts thereof.

H-IdIFPRPGGGGNGDFEEIPEEYL-OHFormula XXIII.

In some embodiments, active agents for use in treating glaucoma include compounds shown in Formula XXIII, and pharmaceutically-acceptable prodrugs, esters and salts thereof, further comprising 1-5 monomers independently selected from Lys, His, Arg, at the N-terminus or the C-terminus. An active agent may further have conservative replacement of 1-5 monomers.

In some embodiments, active agents for use in treating glaucoma include hirudin shown in Formula XXIV, and pharmaceutically-acceptable prodrugs, esters and salts thereof.

### Seq Id No:1

H-NGDFEEIPEEYLA-OHFormula XXIV.

In some embodiments, active agents for use in treating glaucoma include compounds shown in Formula XXIV, and pharmaceutically-acceptable prodrugs, esters and salts thereof, further comprising 1-5 monomers independently selected from Lys, His, Arg, at the N-terminus or the C-terminus. An active agent may further have conservative replacement of 1-5 monomers.

In some embodiments, active agents for use in treating glaucoma include rapastinel shown in Formula XXV, and pharmaceutically-acceptable prodrugs, esters and salts thereof.

### Seq Id No:2

H-TPPT-NH2Formula XXV.

In some embodiments, active agents for use in treating glaucoma include rapastinel TFA.

In some embodiments, active agents for use in treating glaucoma include compounds shown in Formula XXV, and pharmaceutically-acceptable prodrugs, esters and salts thereof, further comprising 1-5 monomers independently selected from Lys, His, Arg, at the N-terminus or the C-terminus. An active agent may further have conservative replacement of 1-5 monomers.

In some embodiments, active agents for use in treating glaucoma include apimostinel shown in Formula XXVI, and pharmaceutically-acceptable prodrugs, esters and salts thereof.

H-TPXₐₐT-NH2Formula XXVI

wherein Xₐₐ is a Proline monomer substituted at the branch carbon, where the substituent can be H.

In some embodiments, active agents for use in treating glaucoma include compounds shown in Formula XXVII, and pharmaceutically-acceptable prodrugs, esters and salts thereof.

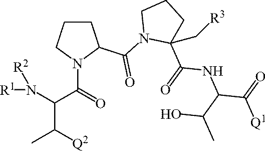

wherein
Q¹, Q² are independently selected from H, hydroxyl, amino, alkoxy, aryloxy, aminoalkoxy;
R¹, R² are independently selected from H, alkyl, cycloalkyl, aryl;
R³ is selected from H, alkyl, aryl, haloalkyl, cycloalkyl, haloaryl, alkylaryl, haloalkylaryl;
and pharmaceutically-acceptable prodrugs, esters and salts thereof.

In some embodiments, active agents for use in treating glaucoma include apimostinel shown in Formula XXVIII, and pharmaceutically-acceptable prodrugs, esters and salts thereof.

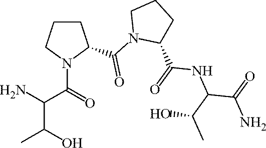 [SMILES: CC(O)C(N)C(=O)N1CCC[C@@H]1C(=O)N1CCC[C@@H]1C(=O)NC(C(N)=O)[C@H](C)O]

which is (2R)-N-((3S)-1-amino-3-hydroxy-1-oxobutan-2-yl)-1-(threonyl-D-prolyl)pyrrolidine-2-carboxamide.

In some embodiments, active agents for use in treating glaucoma include compounds shown in Formula XXVIII, and pharmaceutically-acceptable prodrugs, esters and salts thereof, further comprising 1-5 monomers independently selected from Lys, His, Arg, at the N-terminus or the C-terminus. An active agent may further have conservative replacement of 1-5 monomers.

Embodiments of this invention further contemplate active agents for use in treating glaucoma having a range of structures based on a 9,10-dihydroanthracene.

In some embodiments, active agents for use in treating glaucoma include compounds shown in Formula XXIX.

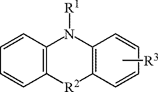

wherein
R¹ is selected from alkyl, cycloalkyl, aminoalkyl, hydroxyalkyl, alkoxyalkyl, aryl, alkenyl, amino-alkenyl, alkynyl, 1,4-piperazinyl, 1-alkyl-1,4-piperazinyl, 1-hydroxyalkyl-1,4-piperazinyl;
R² is selected from C, S, O;
R³ is selected from H, halo, alkyl, amino, —CF₃, —O—CH₃, —S—CH₃;
and pharmaceutically-acceptable prodrugs, esters and salts thereof.

In some embodiments, active agents for use in treating glaucoma include chlorpromazine Formula XXX.

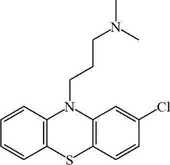 [SMILES: CN(C)CCCN1c2ccccc2Sc2ccc(Cl)cc21]

which is 3-(2-chloro-10H-phenothiazin-10-yl)-N,N-dimethylpropan-1-amine, and pharmaceutically-acceptable prodrugs, esters and salts thereof.

In some embodiments, active agents for use in treating glaucoma include fluphenazine Formula XXXI.

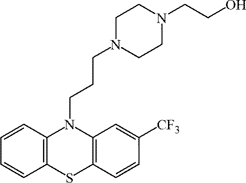 [SMILES: OCCN1CCN(CCCN2c3ccccc3Sc3ccc(C(F)(F)F)cc32)CC1]

which is 2-(4-(3-(2-(trifluoromethyl)-10H-phenothiazin-10-yl)propyl)piperazin-1-yl)ethan-1-ol, and pharmaceutically-acceptable prodrugs, esters and salts thereof.

In some embodiments, active agents for use in treating glaucoma include perphenazine Formula XXXII.

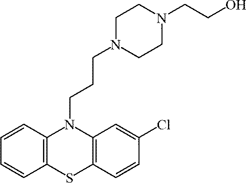 [SMILES: OCCN1CCN(CCCN2c3ccccc3Sc3ccc(Cl)cc32)CC1]

which is 2-(4-(3-(2-chloro-10H-phenothiazin-10-yl)propyl)piperazin-1-yl)ethan-1-ol, and pharmaceutically-acceptable prodrugs, esters and salts thereof.

In some embodiments, active agents for use in treating glaucoma include prochlorperazine Formula XXXIII.

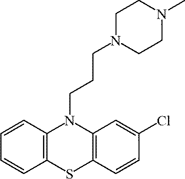 [SMILES: CN1CCN(CCCN2c3ccccc3Sc3ccc(Cl)cc32)CC1]

which is 2-chloro-10-(3-(4-methylpiperazin-1-yl)propyl)-10H-phenothiazine, and pharmaceutically-acceptable prodrugs, esters and salts thereof.

In some embodiments, active agents for use in treating glaucoma include promethazine Formula XXXIV.

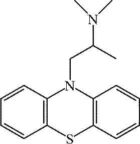 [SMILES: CC(CN1c2ccccc2Sc2ccccc21)N(C)C]

which is N,N-dimethyl-l-(10H-phenothiazin-10-yl)propan-2-amine, and pharmaceutically-acceptable prodrugs, esters and salts thereof.

In some embodiments, active agents for use in treating glaucoma include thioridazine Formula XXXV.

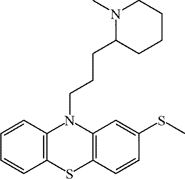 [SMILES: CSc1ccc2c(c1)N(CCCC1CCCCN1C)c1ccccc1S2]

which is 10-(3-(1-methylpiperidin-2-yl)propyl)-2-(methylthio)-10H-phenothiazine, and pharmaceutically-acceptable prodrugs, esters and salts thereof.

In some embodiments, active agents for use in treating glaucoma include trifluoperazine Formula XXXVI.

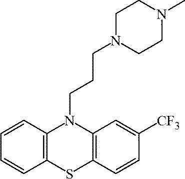 [SMILES: CN1CCN(CCCN2c3ccccc3Sc3ccc(C(F)(F)F)cc32)CC1]

which is 10-(3-(4-methylpiperazin-1-yl)propyl)-2-(trifluoromethyl)-lOH-phenothiazine, and pharmaceutically-acceptable prodrugs, esters and salts thereof.

In some embodiments, active agents for use in treating glaucoma include levomepromazine Formula XXXVII.

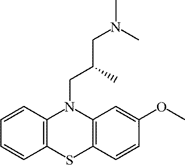 [SMILES: COc1ccc2c(c1)N(C[C@@H](C)CN(C)C)c1ccccc1S2]

which is (S)-3-(2-methoxy-10H-phenothiazin-10-yl)-N,N,2-trimethylpropan-1-amine, and pharmaceutically-acceptable prodrugs, esters and salts thereof.

In some embodiments, active agents for use in treating glaucoma include chlorprothixene Formula XXXVIII.

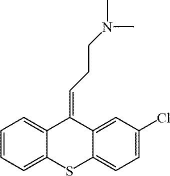 [SMILES: CN(C)CC/C=C1/c2ccccc2Sc2ccc(Cl)cc21]

which is (Z)-3-(2-chloro-9H-thioxanthen-9-ylidene)-N,N-dimethylpropan-1-amine, and pharmaceutically-acceptable prodrugs, esters and salts thereof.

Embodiments of this invention further contemplate active agents for use in treating glaucoma having a range of structures based on a neomycin.

Neomycin can be derived from S. fradiae. Neomycin may be composed of three components A, B, and C. Neomycin may be used in a sulfate salt form.

In some embodiments, active agents for use in treating glaucoma include compounds shown in Formula XXXIX.

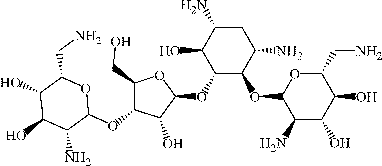

Embodiments of this invention further contemplate active agents for use in treating glaucoma having a range of structures based on a boceprevir.

Boceprevir can be a synthetic tripeptide.

In some embodiments, active agents for use in treating glaucoma include compounds shown in Formula XXXX.

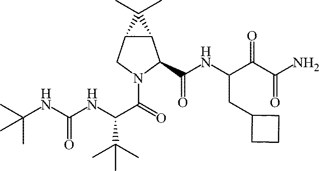 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

Embodiments of this invention further contemplate active agents for use in treating glaucoma having a range of structures based on a levetriacetam.

Levetiracetam can be a synthetic pyrrolidinone and carboxamide that is N-methylpyrrolidin-2-one in which one of the methyl hydrogens is replaced by an aminocarbonyl group, while another is replaced by an ethyl group (the S enantiomer).

In some embodiments, active agents for use in treating glaucoma include compounds shown in Formula XXXXI.

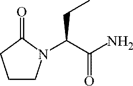

Embodiments of this invention further contemplate active agents for use in treating glaucoma having a range of structures based on a pramiracetam.

Pramiracetam can be a synthetic N-[2-[di(propan-2-yl)amino]ethyl]-2-(2-oxopyrrolidin-1-yl)acetamide.

In some embodiments, active agents for use in treating glaucoma include compounds shown in Formula XXXXII.

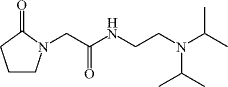 [SMILES: CC(C)N(CCNC(=O)CN1CCCC1=O)C(C)C]

### Active Agent Forms

Embodiments of this invention further contemplate use of active agents for treating glaucoma disorders. In some aspects, a glaucoma disorder may be treated by administering an active agent for affecting EV-complexes. An effective amount an active agent can be administered for ameliorating, alleviating, inhibiting, lessening, delaying, and/or preventing at least one symptom or condition of a glaucoma disorder.

The molecules, compounds and/or compositions of this disclosure may be asymmetric, having one or more chiral stereocenters. A compound containing one or more chiral centers can include substances described as an “isomer,” a “diastereomer,” a “stereoisomer,” an “optical isomer,” an “enantiomer,” or as a “racemic mixture.” Conventions for stereochemical nomenclature, for example the stereoisomer naming rules of Cahn, Ingold and Prelog, as well as methods for the determination of stereochemistry and the separation of stereoisomers are known in the art. See, e.g., March’s Advanced Organic Chemistry (7th ed., 2013). The compounds, composition and structures of this disclosure are intended to encompass all possible isomers, stereoisomers, diastereomers, enantiomers, and/or optical isomers that exist for the compound, composition and/or structure, including any mixture, racemate, or racemic or other mixtures thereof.

A compound can exist in un-solvated and solvated forms, or hydrated forms. In this disclosure, solvated forms, with pharmaceutically acceptable solvents, such as water or ethanol, are to be taken as equivalent to the un-solvated forms. Compounds and salts, or solvates thereof, may also exist in tautomeric forms, which are to be taken as equivalent.

The molecules, compounds and/or compositions of this disclosure may be found in different crystalline forms, which are intended to be encompassed by this disclosure.

Examples of pharmaceutically-acceptable salt forms include ammonium salts, alkali metal salts including sodium, lithium, and potassium salts, alkaline earth metal salts including calcium and magnesium salts, salts with organic bases, for example, organic amines, such as benzathines, dicyclohexylamines, hydrabamines formed with N,N-bis(dehydroabietyl)ethylenediamine), N-methyl-D-glucamines, N-methyl-D-glucamides, t-butyl amines, and salts with amino acids including arginine and lysine.

Examples of pharmaceutically-acceptable forms include esters when amenable to the structure.

Examples of pharmaceutically-acceptable salt forms include include acetates, adipates, alginates, ascorbates, aspartates, benzoates, benzenesulfonates, bisulfates, borates, butyrates, citrates, camphorates, camphorsulfonates, cyclopentanepropionates, hydrochlorides, hydrobromides, hydroiodides, 2-hydroxyethanesulfonates, lactates, maleates, methanesulfonates, 2-napthalenesulfonates, nicotinates, nitrates, oxalates, pectinates, persulfates, digluconates, dodecylsulfates, ethanesulfonates, fumarates, glucoheptanoates, glycerophosphates, hemisulfates, heptanoates, hexanoates, 3-phenylpropionates, phosphates, picrates, pivalates, propionates, salicylates, succinates, sulfates, sulfonates, tartarates, thiocyanates, toluenesulfonates, and undecanoates.

### Compositions and Formulations

An active agent of this disclosure can include drugs and agents for diseases of the eye, including small molecule drugs, peptides, antibodies and protein agents.

A formulation of an active agent may be prepared by dissolving a composition in water to produce an aqueous solution and rendering the solution sterile.

A formulation of this disclosure can be in the form of a sterile injectable aqueous or oily suspension. A suspension can be formulated including a dispersing or wetting agent. A sterile injectable preparation can be a sterile injectable solution or suspension in a non-toxic, pharmaceutically acceptable diluent or solvent.

Examples of solvents include water, water for injection, Ringer’s solution, balanced salt saline, isotonic sodium chloride solution, 1,3-butanediol, synthetic mono-or diglycerides, and fatty acids such as oleic acid.

A formulation of this disclosure can be in the form of eye drops for topical delivery.

An ophthalmic formulation can be a solution or suspension for topical administration. A composition can be a viscous or semi-viscous gel, or other solid or semisolid compositions.

An ophthalmic formulation can be locally delivered by direct injection or by use of an infusion pump.

In some embodiments, an ophthalmic-acceptable formulation may contain an osmolality modulator to adjust the osmolality of the formulation from about 200 to about 500 mOsm/Kg, or from about 250 to about 400 mOsm/Kg, or from about 280 to about 320 mOsm/Kg. Examples of osmolality excipients include dextrose, sodium chloride, potassium chloride, glycerin, and combinations thereof.

An ophthalmic formulation can include artificial tears carriers.

An ophthalmic formulation can include a phospholipid carrier.

An ophthalmic formulation can include a surfactant, a preservative, an antioxidant, a tonicity adjusting excipient, a buffer, a co-solvent, and a viscosity excipient.

An ophthalmic formulation may include an excipient to adjust osmolarity of the formulation.

An ophthalmic formulation can include a viscosity excipient such as a polysaccharide, hyaluronic acid, chondroitin sulfate, a dextran, a cellulose polymer, a vinyl polymer, and an acrylic acid polymer.

An ophthalmic formulation may have a viscosity of from 1 to 400 centipoises, or from 1 to 100 centipoises, or from 2 to 40 cps. An ophthalmic formulation may have a viscosity of about 15, 20, 25, 30, 40, or 50 centipoises.

Examples of excipients or carriers for a formulation of this invention include ophthalmologically acceptable preservatives, viscosity enhancers, penetration enhancers, buffers, sodium chloride, sterile water, water for injection, and combinations thereof.

A dosage form of a composition of this invention can be liquid or an emulsion. A dosage form of the composition of this invention can be solid, which can be reconstituted in a liquid prior to administration.

A composition of this disclosure can also be in the form of an oil-in-water emulsion. The oily phase can be a vegetable oil or a mineral oil.

Examples of emulsifying agents include naturally-occurring gums, gum acacia, gum tragacanth, phosphatides, esters of fatty acids, hexitol, sorbitan monooleate, and polyoxyethylene sorbitan monooleate.

Embodiments of this invention can advantageously provide effective activity of an active agent at dosage levels significantly lower than conventional dosage levels.

An effective amount of an active agent composition of this disclosure can be an amount sufficient to ameliorate or reduce a symptom of the disease treated.

A composition may be administered as a single dosage or may be administered in a regimen with repeated dosing.

An appropriate dosage level of an active agent can be determined by a skilled artisan. In some embodiments, an active agent can be present in a composition in an amount from about 0.001% to about 40%, or from about 0.01 % to about 20%, or from about 0.1% to 10% by weight of the total formulation.

An active agent of this disclosure can be combined with one or more pharmaceutically acceptable carriers. A carrier can be in a variety of forms including fluids, viscous solutions, gels, or solubilized particles. Examples of carriers include pharmaceutically acceptable diluents, solvents, saline, and various buffers.

Some examples of carriers, excipients and additives are given in U.S. Pharmacopeia National Formulary (2014); Handbook of Pharmaceutical Excipients (7th ed., 2013); Handbook of Preservatives (2004, Synapse Information Resources); Remington: The Science and Practice of Pharmacy (22nd ed. 2013); Remington’s Pharmaceutical Sciences (Mack Publishing Co. 1990). Some examples of drugs and delivery are given in Goodman and Gilman, The Pharmacological Basis of Therapeutics (13th ed. 2018, McGraw Hill, NY).

In certain embodiments, an active agent may be delivered without a carrier for reducing extracellular complexes in glaucoma ocular humor.

Examples of carriers include water, pyrogen free water; isotonic saline, Ringer’s solution, ethyl alcohol, and phosphate buffer solution.

A formulation of this disclosure may include a polymer such as a polyethylene glycol (PEG), polypropylene glycol, or poly(lactic-co-glycolic acid) having a molecular weight of about 0.2 to about 50 kDa.

Examples of carrier polymers include polyvinyl acetate, polyvinyl alcohol, polyvinylpyrrolidone, chitosan, collagen, sodium alginate, gelatin, hyaluronic acid, polylactic acid, poly(lactic acid-glycolic acid) copolymer, polyhydroxybutyric acid, poly(hydroxybutyric acid-glycolic acid) copolymer, cellulose, hydroxymethylcellulose, hydroxypropylcellulose, fatty acid esters, and polyglycerins.

Examples of additives include saccharides, sucrose, mannitol, lactose, L-arabinose, D-erythrose, D-ribose, D-xylose, D-mannose, D-galactose, lactulose, cellobiose, gentibiose, glycerin, polyethylene glycol, N-methylpyrrolidone, oligovinyl alcohol, ethanol, ethylene glycol, and propylene glycol.

Examples of solubility enhancing agents include cyclodextrins.

A formulation can include galactose, lactose, mannitol, monosaccharide, fructose, maltose, galactose, glucose, D-mannose, sorbose, disaccharide, lactose, sucrose, trehalose, cellobiose, polysaccharide, maltodextrin, dextran, starch, mannitol, or xylitol.

An ophthalmic formulation may include a lipid such as dipalmitoylethylphosphocholine, dioleoyl phosphatidylethanolamine, or 3B-[N-(N′,N′-Dimethylaminoethane)-carbamoyl] cholesterol.

An ophthalmic formulation may include a lipid such as 1,2-Dioleoyl-sn-Glycero-3-[Phospho-L-Serine], 1,2- Dioleoyl-sn-Glycero-3- Phosphate.

An ophthalmic formulation may include a lipid such as 1,2-Dipalmitoyl-sn-Glycero-3-Phosphocholine, distearoylphosphatidylcholine, diarachidoylphosphatidylcholin, dipalmitoyl phosphatidylethanolamine.

An ophthalmic formulation may include a fatty acid, oleic acid, myristoleic, or aracadonic acid.

An ophthalmic formulation may include a phospholipid such as phosphatidylcholine, lecithin, phosphatidylglycerol, phosphatidylinositol, phosphatidylserine, and phosphatidylethanolamine.

An ophthalmic formulation may include a polymer such as polyvinylpyrrolidone, hydroxymethylcellulose, hydroxyethylcellulose, hydroxypropylmethylcellulose, hydroxyethylstarch, cyclodextrin, 2-hydroxypropyl-β-cyclodextrin, sulfobutylether-P-cyclodextrin, polyethylene glycol, pectin, poly(lactide-co-glycolide), polylactide, polyethylene imine, or poly-L-lysine.

In some embodiments, an ophthalmic formulation may include one or more of a pH adjusting excipient, a buffering excipient, a tonicity excipient, a viscosity excipient, or a wetting excipient. In certain embodiments, an ophthalmic formulation may include an acidifying excipient, a preservative, an antioxidant, a solubilizing excipient, a humectant, or a suspending excipient.

An ophthalmic formulation may include additives, diluents, delivery vehicles, or carrier materials such as a polymer, a polyethylene glycol, a dextran, a diethylaminoethyl dextran, a cyclodextrin, or a carboxymethyl cellulose.

Examples of excipients include sodium chloride, sodium dihydrogen phosphate monohydrate, and disodium hydrogen phosphate anhydrous.

Examples of formulation additives include vegetable oils, olive oil, sesame oil, coconut oil, mineral oil, and paraffin.

Examples of dispersing or wetting agents include lecithin, polyoxyethylene stearate, heptadecaethyleneoxycetanol, polyoxyethylene sorbitol monooleate, and polyethylene sorbitan monooleate.

Examples of antioxidants include ascorbic acid, cysteine hydrochloride, sodium bisulfite, sodium metabisulfite, sodium sulfite, ascorbyl palmitate, butylated hydroxyanisole, butylated hydroxytoluene, lecithin, propyl gallate, alpha-tocopherol, citric acid, ethylenediamine tetraacetic acid, sorbitol, tartaric acid, and phosphoric acid.

Examples of formulation additives include a thickening agent, for example beeswax, paraffin, or cetyl alcohol.

Examples of formulation excipients include a suspending excipient, sodium carboxymethylcellulose, methylcellulose, hydropropyl-methylcellulose, sodium alginate, polyvinylpyrrolidone, gum tragacanth, or gum acacia.

An ophthalmic formulation may include a carrier or co-solvent such as Polysorbate 20, 60 or 80, Pluronic F-68, F-84 or P-103, Tyloxapol, Cremophor, sodium dodecyl sulfate, glycerol, PEG 400, propylene glycol, cyclodextrin, and combinations thereof. A carrier or co-solvent can be used in concentrations from about 0.01% to about 2% by weight.

An ophthalmic formulation may include a gel excipient such as gellan, xanthan gum, and combinations thereof.

An ophthalmic formulation may include a viscosity enhancer such as polyvinyl alcohol, methyl cellulose, hydroxy propyl carboxymethyl cellulose, hydroxymethylcellulose, hydroxyethylcellulose, hydroxypropylmethylcellulose, methylcellulose, polyvinylpyrrolidone, and combinations thereof. A viscosity enhancer can be used in concentrations from about 0.01% to about 2% by weight.

An ophthalmic formulation may include a preservative such as benzalkonium chloride, chlorobutanol, benzododecinium bromide, methyl paraben, propyl paraben, phenylethyl alcohol, edetate disodium, sorbic acid, onamer, polyquaternium-1, hydroxybenzoate, sodium benzoate, phenol, cresol, p-chloro-m-cresol, benzyl alcohol, thimerosal, sorbic acid, benzethonium chloride, and combinations thereof. A preservative can be used in concentrations from about 0.001% to about 1.0% by weight.

A unit dose composition can be sterile, but may not contain a preservative.

An ophthalmic formulation may include a pH adjusting excipient such as citric acid buffer, acetic acid buffer, succinic acid buffer, malic acid buffer, and gluconic acid buffer.

An ophthalmic formulation may include an additional acid such as hydrochloric acid, or and additional base, such as sodium hydroxide for pH adjustment.

Examples of pH control agents include arginine, sodium hydroxide, glycine, hydrochloric acid, and citric acid.

An ophthalmic formulation may include a buffer such as citric acid, ascorbic acid, gluconic acid, carbonic acid, tartaric acid, succinic acid, acetic acid, phthalic acid, tris, tromethamine hydrochloride, and phosphate buffer.

An ophthalmic formulation may include a surfactant.

Examples of a surfactant include nonionic surfactants, polysorbate-80, polysorbate-20, polysorbates, sorbitan esters, a lipid, a phospholipid, lecithin, a phosphatidylcholine, a phosphatidylethanolamine, a phosphatidylglycerol, a fatty acid, a fatty ester, a cholesterol.

Examples of surfactants include oleic acid, sorbitan trioleate, and long chain diglycerides.

Examples of surfactants include beractant, poractant alfa, and calfactant.

An ophthalmic formulation may include a tonicifier tonicity adjusting excipient.

Examples of a tonicity adjusting excipient, isotonizing excipient, include sodium chloride, mannitol, and sorbitol.

Examples of a tonicity adjusting excipient include sugars, polyols, amino acids, and organic and inorganic salts.

Embodiments of this invention include kits containing any of reagents, pharmaceutical excipients, active agents, and instructions for use.

A kit may include a container or formulation that contains one or more active agents formulated in a pharmaceutical preparation for delivery. An ophthalmic formulation kit can be a multidose form.

A kit may include a dispenser or dropping device for topical delivery and use.

A kit can include one or more unit doses of a composition for delivery. A unit dose can be hermetically sealed to preserve sterility.

### Use of Agents for Glaucoma

In further embodiments, a composition of this disclosure can be administered locally. A composition may be administered locally to ocular tissue. As used herein, the term ocular tissue refers to the eye, including tissues within the conjunctiva and or sclera, e.g., the retina, and outside the sclera, e.g., ocular muscles within the orbit. Ocular tissue also includes tissues neurologically connected to, but distinct from the eye, such as the optic nerve, the geniculate nucleus and the visual cortex. Local administration to ocular tissue can be achieved via extraocular topical eye drops, or intraocular administration. Intraocular administration can be carried out via intracameral administration, intravitreal administration, or subretinal administration.

In some embodiments, a composition of this disclosure can be administered extraocular. Extraocular administration can be achieved via topical eye drops.

In some embodiments, a composition of this disclosure can be administered intraocularly. Intraocular administration can be achieved via intracameral administration, intravitreal administration, or subretinal administration.

In some embodiments, a composition of this disclosure can be administered systemically. Systemic administration can be achieved via intravenous administration, oral administration, intraarterial administration, inhalation, intranasal administration, intraperitoneal administration, intra-abdominal administration, subcutaneous administration, intra-articular administration, intrathecal administration, transdural administration, transdermal administration, submucosal administration, sublingual administration, enteral administration, parenteral administration, percutaneous administration, periarticular administration, or intraventricular administration.

In additional embodiments, local administration to ocular tissue can be achieved via periocular administration. Periocular administration can be carried out via subconjunctival injection, sub-Tenon’s injection, direct periocular injection, or depot periocular injection.

A subject may be administered a therapeutically effective amount of the composition. A therapeutically effective amount can be an amount effective to ameliorate, alleviate, inhibit, lessen, delay, and/or prevent at least one symptom or condition of the condition being treated.

In certain embodiments, a therapeutically effective amount can be the amount effective to ameliorate the ocular condition being treated. The dose may be determined according to various parameters, especially according to the severity of the condition, age, and weight of the patient to be treated; the route of administration; and the required regimen. A physician will be able to determine the required route of administration and dosage for any particular patient. Dosages may vary depending on the relative potency of the composition being administered, and can generally be estimated based on the half maximal effective concentration (EC50) found to be effective in in vitro and in vivo models.

Embodiments of this invention further contemplate processes for making the agents of this disclosure. Methods known in the art, with suitable modifications, can be used. Some examples are given in Greene, Protective Groups in Organic Synthesis (1999), March’s Advanced Organic Chemistry (7th ed., 2013).

### Pharmaceutical Forms

Some compounds are described in Goodman and Gilman, The Pharmacological Basis of Therapeutics (1996, 9th Ed).

As used herein, the term “pharmaceutically acceptable salt” can refer to a salt of a compound that does not adversely affect an organism and maintains the biological and/or pharmaceutical activity of the compound.

Examples of a pharmaceutically acceptable salt include acid addition salts of a compound.

Examples of a pharmaceutically acceptable salt include those obtained by reacting a compound with inorganic acids such as hydrohalic acid, such as a hydrochloric acid or hydrobromic acid, a sulfuric acid, a nitric acid or a phosphoric acid.

Examples of a pharmaceutically acceptable salt include those obtained by reacting a compound with an organic acid such as an aliphatic or aromatic carboxylic or sulfonic acid, for example, a formic acid, an acetic acid, a succinic acid, a lactic acid, a malic acid, a tartaric acid, a citric acid, an ascorbic acid, a nicotinic acid, a methanesulfonic acid, an ethanesulfonic acid, a p-toluenesulfonic acid, a salicylic acid, or a naphthalene sulfonic acid.

Examples of a pharmaceutically acceptable salt include those obtained by reacting a compound with a base to form a salt such as an ammonium salt, an alkali metal salt, a sodium salt, a potassium salt, an alkaline earth metal salt, a calcium salt, a magnesium salt, or a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine and lysine.

### Chemical Groups

As used herein, a C(14-24)alkenyl group may be any one of C(14:1(5))alkenyl, C(14:1(9))alkenyl, C(16:1(7))alkenyl, C(16:1(9))alkenyl, C(18:1(3))alkenyl, C(18:1(5))alkenyl, C(18:1(7))alkenyl, C(18:1(9))alkenyl, C(18:1(11))alkenyl, C(18:1(12))alkenyl, C(18:2(9,12))alkenyl, C(18:2(9,11))alkenyl, C(18:3(9,12,15))alkenyl, C(18:3(6,9,12))alkenyl, C(18:3(9,11,13))alkenyl, C(18:4(6,9,12,15))alkenyl, C(18:4(9,11,13,15))alkenyl, C(20:1(9))alkenyl, C(20:1(11))alkenyl, C(20:2(8,11))alkenyl, C(20:2(5,8))alkenyl, C(20:2(11,14))alkenyl, C(20:3(5,8,11))alkenyl, C(20:4(5,8,11,14))alkenyl, C(20:4(7,10,13,16))alkenyl, C(20:5(5,8,11,14,17))alkenyl, C(20:6(4,7,10,13,16,19))alkenyl, C(22:1(9))alkenyl, C(22:1(13))alkenyl, and C(24:1(9))alkenyl.

As used herein, the term alkyl refers to a hydrocarbyl radical of a saturated aliphatic group, which can be of any length unless otherwise specified. An alkyl group can be a branched or unbranched, substituted or unsubstituted aliphatic group containing from 1 to 24 carbon atoms. This definition also applies to the alkyl portion of other groups such as, for example, cycloalkyl, alkoxy, alkanoyl, and aralkyl, for example.

Examples of alkyl groups include C(1-4)alkyl, which includes methyl, ethyl, propyl, iso-propyl, n-butyl, iso-butyl, sec-butyl and t-butyl.

As used herein, an alkyl group refers to a C1-24 alkyl group, more preferably a C1-12 alkyl group, yet more preferably a C1-8 alkyl group, and even more preferably a C1-4 alkyl group.

As used herein, the term alkenyl refers to a hydrocarbyl radical having at least one carbon-carbon double bond. An alkenyl group can be a branched or unbranched, substituted or unsubstituted hydrocarbyl radical having 2 to 24 carbon atoms and at least one carbon-carbon double bond. An alkenyl group has one or more carbon-carbon double bonds.

As used herein, an alkenyl group refers to a C2-24 alkenyl group, more preferably a C2-12 alkenyl group, yet more preferably a C2-8 alkenyl group, and even more preferably a C2-4 alkenyl group.

As used herein, the term substituted refers to an atom having one or more substitutions or substituents which can be the same or different and may include a hydrogen substituent. Thus, the terms alkyl, cycloalkyl, alkenyl, alkoxy, and aryl, for example, refer to groups which can include substituted variations. Substituted variations include linear, branched, and cyclic variations, and groups having a substituent or substituents replacing one or more hydrogens attached to any carbon atom of the group.

Examples of substituents and substituted groups include alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

### Devices and Systems

This invention further provides a microfluidic device and system for measuring pressure in a fluid.

In some aspects, a microfluidic device and system of this invention can be used for measuring ocular fluid influence on IOP.

In additional aspects, a microfluidic device and system of this invention can be used for determining the ability of a substance to reduce IOP in ocular fluids.

In further aspects, a microfluidic device and system of this invention can be used for determining the ability of a substance to increase ocular fluid outflows.

In certain aspects, a microfluidic device and system of this invention can be used for diagnosing the appearance of symptoms of an ocular disease.

In further aspects, a microfluidic device and system of this invention can be used for diagnosis of disease, including cancers, glaucoma, hypertension, rheumatological diseases, and other aggregating diseases.

In further aspects, a microfluidic device and system of this invention can be used for purifying aggregate particles, wherein smaller particles may be collected at one end, and larger particles may be collected at a different other end by reversal of flow.

In further aspects, a microfluidic device and system of this invention can be used for purifying or separating particles greater than 1 µm in diameter from particles less than 1 µm, or separating particles greater than 2 µm in diameter from particles less than 2 µm, or separating particles greater than 3 µm in diameter from particles less than 3 µm.

In further aspects, a microfluidic device and system of this invention can be used for measuring the relative viscosity and flow properties of biological and clinical fluids.

A microfluidic device and system of this invention may comprise a microfluidic chip that can be held in a substrate.

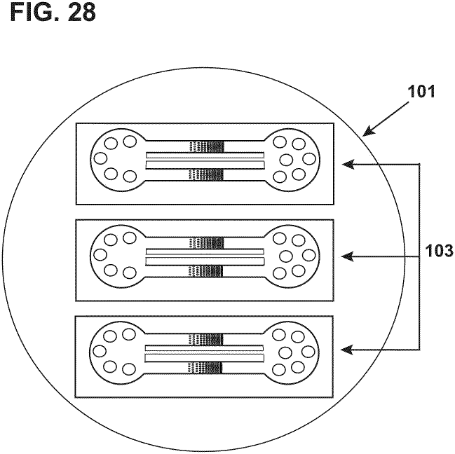

FIG. 28 shows a plan view of a microfluidic chip embodiment of this invention. In this format, a silicon wafer master 101 is printed with three microfluidic channel chip patterns 103. A silicon wafer 101 can be used as a substrate. Photoresist can be poured onto the substrate and exposed to UV light, which forms the pattern of the microfluidic chips 103. Together, the wafer and photoresist form a mold onto which PDMS can be poured. Once set, the PDMS can be peeled off the mold, giving three casts of microfluidic chips per wafer. These casts can be adhered to glass slides to form the final microfluidic chips.

A microfluidic chip of this invention can have a channel for restricted flow of a fluid, and an inlet and an outlet for fluid flow. A pump may be used to apply head pressure of a fluid at the inlet. In some embodiments, a reduced or vacuum pressure can be used at the outlet to adjust flow.

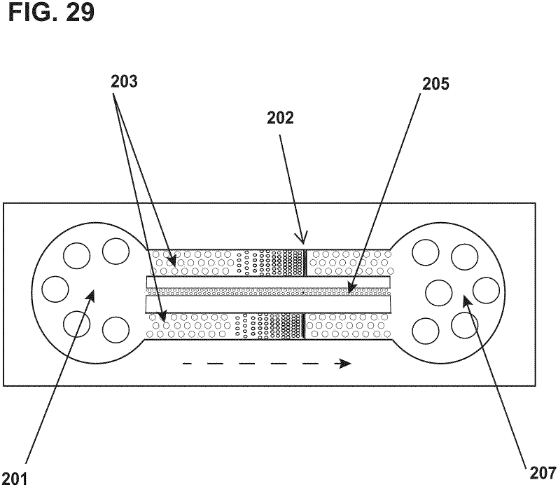

FIG. 29 shows a plan view of a microfluidic chip insert in an embodiment of a device of this invention. The chip has two restriction channels 203, in this example each 2500 um wide and 25,000 um in length. The restriction channels 203 contain pillars of various diameters and spacing, shown by circles. The chip has a third uniform flow channel 205 having pillars of uniform size and spacing which do not significantly restrict the flow. The chip has an inlet reservoir 201 and an outlet reservoir 207, which also contain larger pillars. The dashed arrow shows the direction of flow from the inlet reservoir towards the outlet reservoir.

A microfluidic chip of this invention can have one or more channels for restricted flow of a fluid, and one or more uniform or continuous flow channels. In some embodiments, the uniform flow channel does not present a restriction to fluid flow in the channel. The uniform continuous flow channel may contain blunt obstructions for creating turbulent flow and/or a tortuous path for flowing fluid.

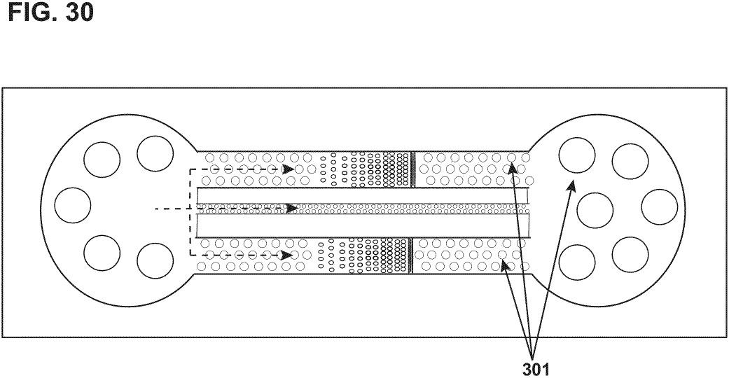

FIG. 30 shows a plan view corresponding to FIG. 29. FIG. 30 shows PDMS polymeric pillars 301 of various sizes represented by circles. The flow of biofluid through three channels is shown by dashed arrows.

In certain embodiments, blunt or non-blunt obstructions may be provided in a restriction fluid channel to create a tortuous or vortex pattern of flow in certain regions.

In certain embodiments, the blunt obstructions of a restriction channel may provide a Reynolds number of greater than 500, or greater than 1000, or greater than 10,000, or greater.

In additional embodiments, a continuous flow channel may be located in between various restriction channels.

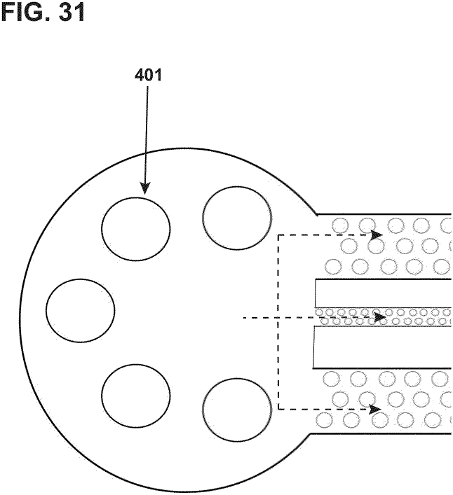

FIG. 31 shows a plan view corresponding to the inlet reservoir of FIG. 29. FIG. 31 shows pillars 401 represented by circles. The flow of biofluid through three channels is shown by dashed arrows.

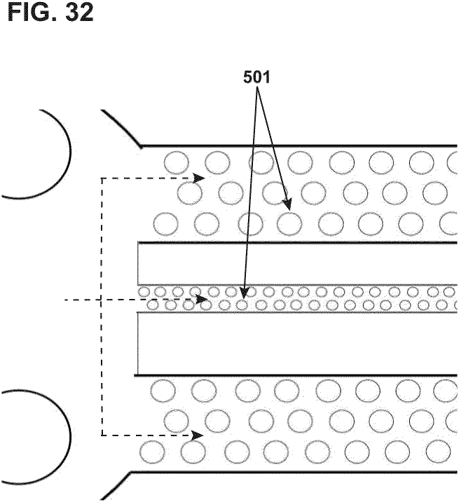

FIG. 32 shows a plan view corresponding to the inlet reservoir region of FIG. 29. FIG. 32 shows pillars 501 represented by circles. The flow of biofluid through three channels is shown by dashed arrows.

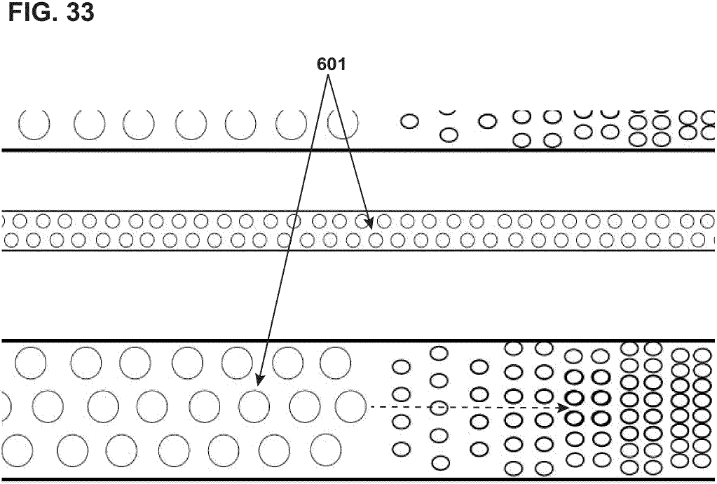

FIG. 33 shows a plan view corresponding to the channel region of FIG. 29. FIG. 33 shows pillars 601 represented by circles. The flow of biofluid through a channel is shown by a dashed arrow. The microfluidic channel device of this invention has regions of different spacing and/or size of pillars or obstructions creating turbulent or restricted flow.

In certain embodiments, a microfluidic channel device of this disclosure may have regions simulating an ocular trabecular mesh.

A device of this invention may include a meshwork composition which contains extracellular matrix bodies or complexes. Extracellular matrix bodies or complexes for use in a meshwork composition may be extracted or purified from glaucoma ocular humor. The ocular humor may be from animal or clinical sources.

In further embodiments, a microfluidic chip of this invention can have a one or more channels for restricted flow of a fluid and one or more uniform flow channels. The uniform flow channels may contain blunt obstructions for creating turbulent flow and/or a tortuous path for flowing fluid.

In further embodiments, a microfluidic chip of this invention can have a 1-20 channels for restricted flow of a fluid and 1-10 uniform flow channels, arranged in any order on a substrate. The uniform flow channels may be distributed in any manner with respect to the restricted flow channels.

In certain embodiments, uniform flow channels may alternate in co-linear or parallel positions with respect to restricted flow channels. In additional embodiments, uniform flow channels may be above or below restricted flow channels. In some embodiments, uniform flow channels may be arranged in a separate substrate from a chip that contains restriction flow channels.

In further embodiments, the uniform flow channels may provide fluid communication from an inlet reservoir to an outlet reservoir. In certain embodiments, a uniform flow channel may provide fluid communication from an outlet reservoir to the source of the fluid entering an inlet reservoir.

In certain embodiments, the total cross sectional area of uniform flow channels may be greater than, or less than the total cross sectional area of restriction flow channels in a microfluidic device of this invention. In various embodiments, uniform flow channels may not contain obstructions and may not have tortuous fluid flow. In such embodiments, uniform flow channels can have laminar or turbulent fluid flow.

A microfluidic chip of this invention can have one or more restriction channels for restricted flow of a fluid. The restricted flow may be due to various arrangements of blunt or non-blunt obstructions or pillars in the channel. In some embodiments, the pillars may present a shape to the flowing fluid, such as circular, spherical, triangular, square, polygonal, diamond, fin-shaped, and combinations thereof.

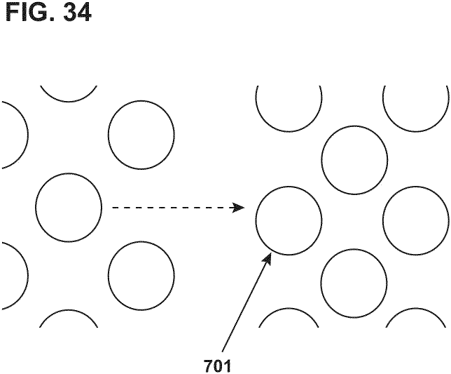

FIG. 34 shows an expanded plan view corresponding to the channel region of FIG. 29. FIG. 34 shows pillars 701 represented by circles. The flow of biofluid through a channel is shown by a dashed arrow. This view shows a transition from 50 um gaps between pillars to 25 um gaps in a restriction channel.

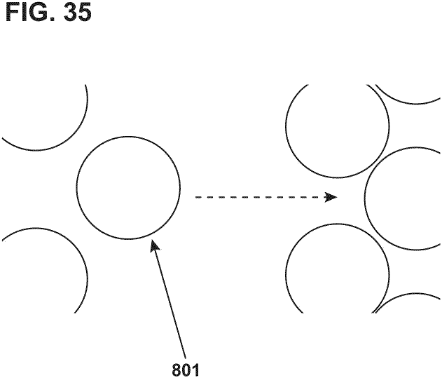

FIG. 35 shows an expanded plan view corresponding to the channel region of FIG. 29. FIG. 35 shows pillars 801 represented by circles. The flow of biofluid through a channel is shown by a dashed arrow. This view shows a transition from larger to smaller gaps between pillars in a restriction channel.

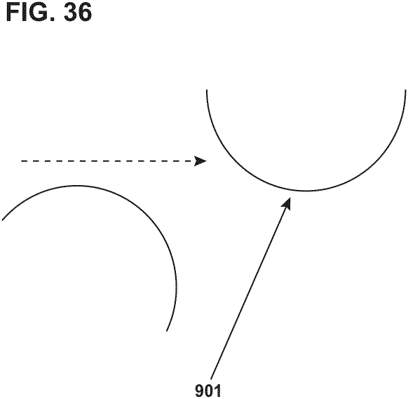

FIG. 36 shows an expanded plan view corresponding to the channel region of FIG. 29. FIG. 36 shows pillars 901 represented by circles. The flow of biofluid through a channel is shown by a dashed arrow.

In further embodiments, restricted flow in a channel may be due to various arrangements of blunt or non-blunt obstructions or pillars in the channel, where the size and spacing of obstructions changes with distance along the channel.

In certain embodiments, the size and/or spacing of blunt or non-blunt obstructions or pillars in a restriction channel may change with distance along the channel. The size and/or spacing of blunt or non-blunt obstructions may reduce with distance along the channel. At some position in a restriction channel, the size and/or spacing of blunt or non-blunt obstructions may be reduced to a level which provides a maximal restriction barrier to flow.

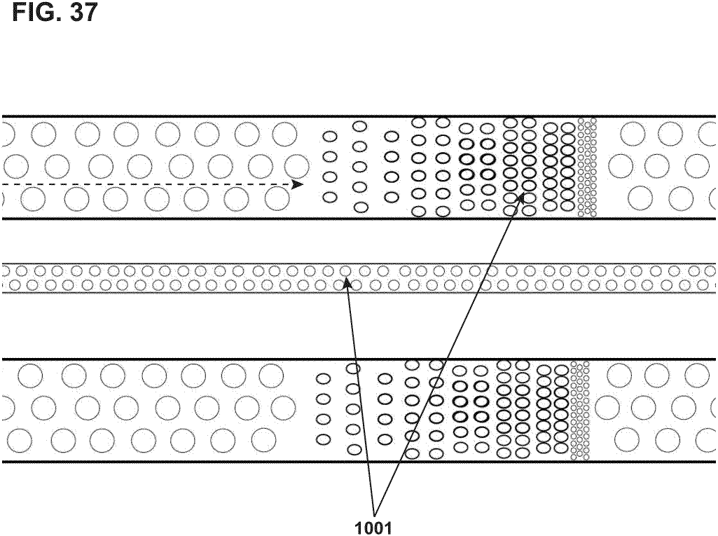

FIG. 37 shows an expanded plan view corresponding to the channel region of FIG. 29. FIG. 37 shows pillars 1001 represented by circles. The flow of biofluid through a channel is shown by a dashed arrow. This view shows channels having regions of blunt pillar obstructions 1001 which can create turbulent flow.

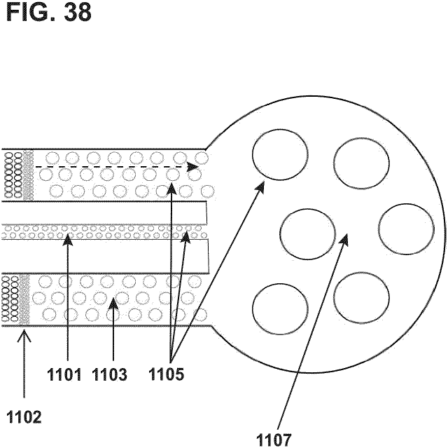

FIG. 38 shows an expanded plan view corresponding to the outlet reservoir 1107 of FIG. 29. FIG. 38 shows pillars 1101, 1103, and 1105 of various sizes. The flow of biofluid through a channel is shown by a dashed arrow. In this embodiment, the outer restriction channels each contain a barrier 1102 formed by very small and closely-spaced pillars.

In further embodiments, various arrangement of blunt or non-blunt obstructions or pillars in a restriction channel can be used to restrict flow to any level. A wide range of spacings and/or patterns of blunt and/or non-blunt obstructions can be used in a restriction channel. A fluid may have a tortuous path in a restriction flow channel. The spacing of obstructions in a restriction channel and/or the tortuosity of the fluid path can increase with distance along the channel in the direction of flow.

Fluid effluents from the channels of a microfluidic chip of this invention can be collected in an outlet reservoir at the outlet end of the channels. The inflow or insertion of fluid to the channels of a microfluidic chip of this invention can be achieved with a reservoir at the inlet end of the channels.

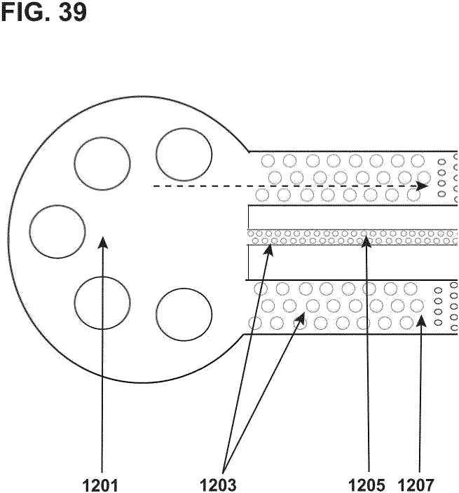

FIG. 39 shows an expanded plan view corresponding to the inlet reservoir 1201 of FIG. 29. FIG. 39 shows pillars 1203 of various sizes. Outer restriction channel 1207 contains pillars of varying size and spacing. Uniform flow channel 1205 contains pillars of uniform size and spacing. The direction of flow of biofluid through an outer channel is shown by a dashed arrow.

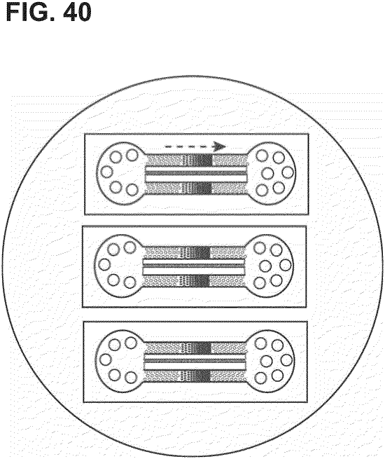

FIG. 40 shows a plan view of a microfluidic chip in an embodiment of a device of this invention. Three microfluidic inserts are shown. The direction of flow of biofluid is shown by a dashed arrow.

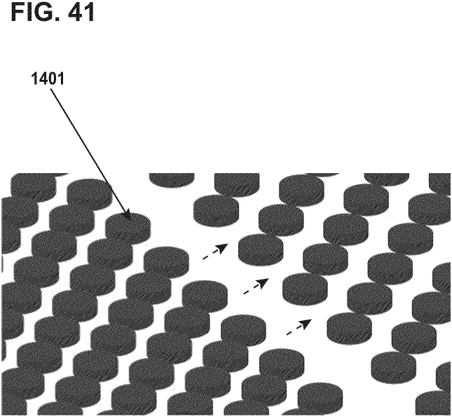

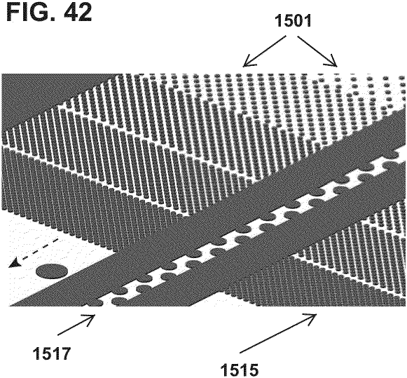

FIG. 41 shows a perspective view of an embodiment of a microfluidic channel device of this invention having blunt pillar obstructions 1401 to flow. FIG. 41 is an expansion of FIG. 42. The direction of flow of biofluid is shown by dashed arrows.

FIG. 42 shows a perspective view of an embodiment of a microfluidic channel device of this invention. FIG. 42 shows a view corresponding to the channel region of FIG. 29. FIG. 42 shows blunt pillar obstructions 1501 of varying spacing in a restriction channel. In this embodiment, a restriction channel can have pillar obstructions 1501 organized in bands of varying spacing between the pillars. The direction of flow of biofluid is shown by a dashed arrow.

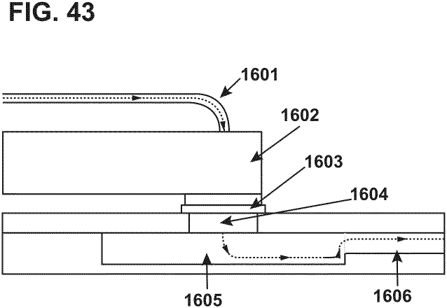

FIG. 43 shows an elevation side view of a microfluidic chip embodiment of this invention. The inlet reservoir 1605 is in fluid communication with a fluid line 1601 for introducing biofluid and/or other fluid into the reservoir. The fluid line 1601 passes through a probe 1602, probe adapter 1603, and hole 1604 defined in a glass cover slide. The biofluid passes through the inlet reservoir 1605 to reach the microfluidic channel 1606. The direction of flow of biofluid is shown by a dashed arrow.

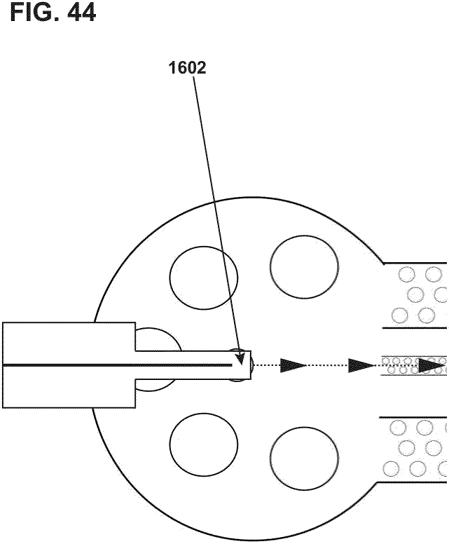

FIG. 44 shows an expanded plan view corresponding to the inlet region of FIG. 29, and the position of a probe 1602 of FIG. 43. The direction of flow of biofluid is shown by a dashed arrow.

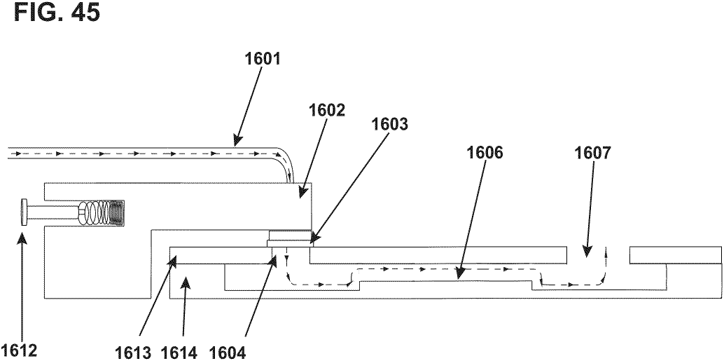

FIG. 45 shows an elevation side view of a microfluidic chip 1614 embodiment of this invention. The inlet reservoir is in fluid communication with a fluid line 1601 for introducing biofluid into the reservoir. The fluid line 1601 passes through a probe 1602, probe adapter 1603, and hole 1604 defined in a glass cover slide 1613. The biofluid passes through the inlet reservoir to reach the microfluidic channel 1606 and flow to the outlet reservoir 1607. A probe adjuster 1612 can be provided to adjust the height of the probe 1602 to create a good seal with the probe adapter 1603 and hole 1604. The direction of flow of biofluid is shown by a dashed arrow.

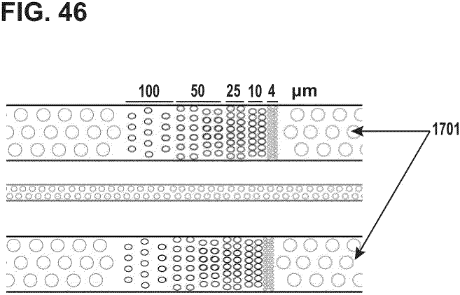

FIG. 46 shows an expanded plan view corresponding to the channel region of FIG. 29. FIG. 46 shows pillars 1701 represented by circles. For this embodiment, some representative lengths of regions of pillar bands in the outer channel are shown in micrometers.

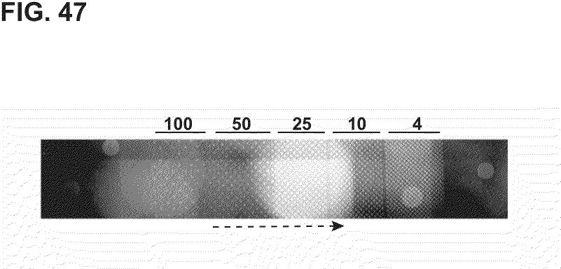

FIG. 47 shows a micrograph of an expanded plan view corresponding to the channel region of FIG. 29. FIG. 47 shows pillars as dots. For this embodiment, some representative lengths of regions of pillar bands in the outer channel are shown in micrometers. The direction of flow of biofluid is shown by a dashed arrow.

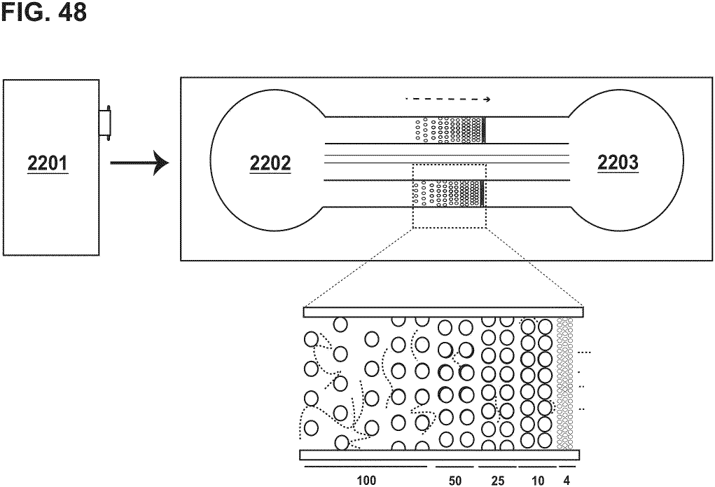

FIG. 48 shows a plan view of an embodiment of a microfluidic device corresponding to FIG. 29. Biofluid can be introduced with a delivery probe 2201 to the inlet region reservoir 2202. The direction of flow of biofluid to the outlet reservoir region 2203 is shown by a dashed arrow. An expansion view for this embodiment shows some representative lengths of regions of pillar bands in the outer channel in micrometers. For this embodiment, dotted lines in the expansion view show possible tortuous paths of biofluid amongst the obstructions.

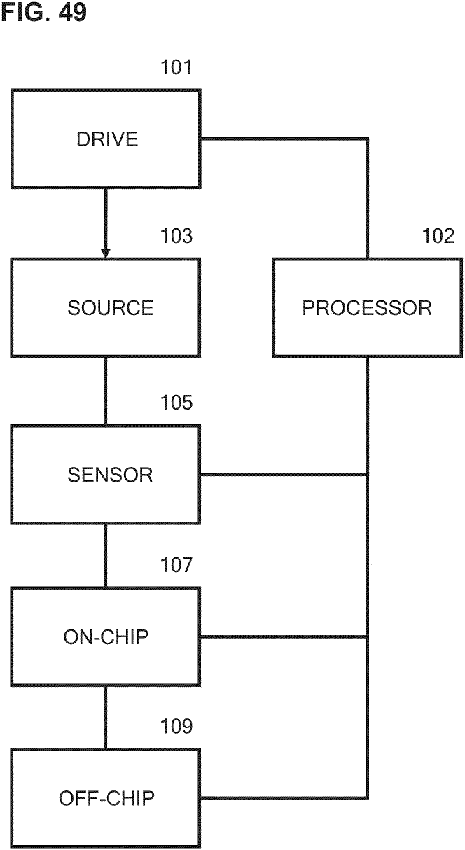

FIG. 49 shows an embodiment of a microfluidic system of this invention. A processor 102 can send control signals and/or receive signals from a fluid drive unit 101, which provides a drive fluid, such as a compressed gas, to a fluid source unit 103. The fluid source unit 103 can contain a fluid, biofluid, carrier, and/or reagents of interest. The fluid, biofluid, carrier, and/or reagents of interest can flow to a sensor unit 105, which can monitor flow rate and/or pressure of the fluid. The fluid, biofluid, carrier, and/or reagents of interest can flow to an on-chip unit 107, which may include a microfluidic device of this invention. The fluid, biofluid, carrier, and/or reagents of interest can enter the inlet reservoir of a microfluidic chip of this invention in the on-chip unit 107. The fluid, biofluid, carrier, and/or reagents of interest can reach the outlet reservoir of a microfluidic chip of this invention in the on-chip unit 107 and flow to an off-chip unit 109. The processor 102 can receive data from the sensor unit 105, and record the flow and/or pressure. The on-chip unit 107 can include analytical tools such as irradiation and light detectors for spectrometry. The off-chip unit 109 can include various analytical tools such as microscopy tools, imagers, and analyzers, chromatography analyzers, mass spectrometry analyzers, and/or magnetic resonance analyzers. The processor 102 can send control signals and/or receive data from the on-chip unit 107 and off-chip unit 109.

In some embodiments, a system or device of this invention can be used to characterize the activity of a biologically active agent toward glaucoma. A system or device of this invention can be used to detect or characterize ocular conditions or parameters in a model system or in patient pathology.

In some aspects, a fluid composition in a system or device of this invention can be analyzed by various techniques. For example, a fluid composition can be analyzed by an imaging technique.

Examples of imaging techniques include electron microscopy, stereoscopic microscopy, wide-field microscopy, polarizing microscopy, phase contrast microscopy, multiphoton microscopy, differential interference contrast microscopy, fluorescence microscopy, laser scanning confocal microscopy, multiphoton excitation microscopy, ray microscopy, and ultrasonic microscopy.

Examples of imaging techniques include positron emission tomography, computerized tomography, and magnetic resonance imaging.

Examples of assay techniques include colorimetric assay, chemiluminescence assay, spectrophotometry, immunofluorescence assay, and light scattering.

In some embodiments, this invention can provide a device for measuring pressure and flow rate of a fluid composition. In certain embodiments, a device can have a meshwork composition lodged in the channel for providing resistance to flow. The meshwork composition may have any one or more of a uveal meshwork, a corneoscleral meshwork, and a juxtacanalicular meshwork. Such meshworks can be simulated with obstructions in a restriction channel, for example, or provided from extraction of ocular humor, bodily fluid, or clinical samples.

Extracellular matrix bodies or complexes for use in a meshwork composition may be composed of various biomolecules or complexed particles, and may have diameters ranging from about 0.5 to about 5,000, or from 0.5 to 1,000, or from 1 to 200, or from 1 to 100, or from 1 to 50, or from 1 to 25, or from 1 to 10, or from 1 to 5 micrometers.

In some embodiments, a meshwork composition can be composed of glass beads, micro beads, magnetic beads, gel particles, dextran particles, or polymer particles. A meshwork composition may also be composed of glass fibers, polymeric fibers, inorganic fibers, organic fibers, or metal fibers.

In certain embodiments, a uveal meshwork or restriction channel may have fenestrations of about 25 micrometers. A corneoscleral meshwork or restriction channel may have fenestrations of about 2-15 micrometers. A juxtacanalicular meshwork or restriction channel may have fenestrations of about 1 to 4 micrometers or less.

A device may further include a fluid reservoir for holding a fluid composition, so that the fluid reservoir is in fluid communication with the inlet of a channel for introducing the fluid composition into the inlet of the channel.

A device of this disclosure can have a drive or pressure source for applying pressure to a drive fluid composition. The drive fluid can enter a fluid reservoir for driving the fluid composition into the inlet of a microfluidic channel.

A device of this invention can have a sensor unit in fluid communication with the fluid composition for measuring the flow rate and pressure of the fluid composition at the inlet of the channel and transmitting the flow rate and pressure to a processor.

Signals and data from units of the system device can be received by a processor. The processor can display the flow rate and pressure. Memory or media can store instructions or files, such as a machine-readable storage medium. A machine-readable storage medium can be non-transitory.

A processor of this disclosure can be a general purpose or special purpose computer. A processor can execute instructions stored in a machine readable storage device or medium. A processor can include an integrated circuit chip, a microprocessor, a controller, a digital signal processor, any of which can be used to receive and/or transmit data and execute stored instructions. A processor can also perform calculations and transform data, and/or store data in a memory, media or a file. A processor may receive and execute instructions which may include performing one or more steps of a method of this invention. A device of this invention can include one or more non-transitory machine-readable storage media, one or more processors, one or more memory devices, and/or one or more user interfaces. A processor may have an integral display for displaying data or transformed data.

In some aspects, a system of this disclosure may have a device having microfluidic channels. One or more channels can be arranged in a microfluidic chip.

A system of this disclosure can include an on-chip unit having one or more detectors for analyzing the fluid composition within the channels or at the inlet or exiting the outlet of the channel. Detectors can also be arranged to detect the fluid composition within the channel.

A system of this disclosure can include an off-chip unit having one or more detectors for analyzing a fluid composition extracted from microfluidic channels.

In certain embodiments, extracellular matrix bodies or complexes for use in a meshwork composition in a system or device of this disclosure may include a fixative, a stabilizing component, or a cross linking component which can transform the structure to a stable, uniform composition.

Examples of stabilizing components include fixatives as described herein, cross linking compounds as described herein, organic solvents, polypeptides, and pharmaceutically-acceptable organic salts.

Extracellular matrix bodies or complexes that are cross linked can be reversibly cross linked, or non-reversibly cross linked.

In some embodiments, a device of this invention may contain extracellular matrix bodies or complexes as a meshwork composition that can be used for identifying or screening active agents for effects in reducing IOP and/or increasing ocular outflows. A meshwork composition may include a drug delivery excipient.

In additional embodiments, a device of this invention may be used for measuring the quantity or level of extracellular matrix bodies or complexes in a test sample. Measuring the quantity or level of extracellular matrix bodies or complexes in a test sample can provide a diagnostic marker level for the test sample. A device of this invention can be used to identify glaucoma or pre-glaucoma in a subject.

In further embodiments, a device of this invention may be used for measuring a pressure which can be related to a quantity or level of extracellular matrix bodies or complexes in a test sample. A pressure value in a channel can be related directly to a quantity or level of extracellular matrix bodies or complexes in a test sample.

In certain embodiments, a device of this invention may be used for measuring an assay value which can be related to a quantity or level of extracellular matrix bodies or complexes in a test sample. An assay value of a composition in a channel can be related directly to a quantity or level of extracellular matrix bodies or complexes in a test sample.

Example of an assay include a colorimetric assay, a chemiluminescence assay, a spectrophotometry assay, an immunoassay, or a light scattering assay.

In some aspects, an aqueous humor or bodily fluid sample from a subject can be provided and analyzed for a quantity of glaucoma extracellular matrix bodies or complexes. The subject can be identified as having glaucoma or pre-glaucoma based on the quantity exceeding a reference value. A reference value can be a quantity or level of glaucoma extracellular matrix bodies or complexes in a reference population of healthy individuals. The subject can be diagnosed as having glaucoma or pre-glaucoma. Subsequent test samples from the subject can be used to monitor a quantity or level of glaucoma extracellular matrix bodies or complexes exceeding or not exceeding a previous test sample, which can be related to reducing IOP and/or increasing ocular outflows in the subject.

In certain embodiments, a quantity or level of glaucoma extracellular matrix bodies or complexes may include one or more of the number, size, density, morphology, and spatial distribution of the extracellular matrix bodies or complexes.

In some embodiments, a reference value can be a quantity or level of glaucoma extracellular matrix bodies or complexes in a reference population of healthy individuals. The reference value can be the average value in samples from the reference population.

Glaucoma may be found in a subject where a test sample from the subject contains a quantity or level of glaucoma extracellular matrix bodies or complexes exceeding a glaucoma reference value.

In certain embodiments, a glaucoma reference value can be that the number of extracellular matrix bodies or complexes per unit sample.

In additional aspects, a meshwork composition in a device of this invention can be an anterior half or portion of an animal eye with lens, wherein the TM of the eye is oriented in between the inlet and the outlet of the channel.

All publications including patents, patent application publications, and nonpatent publications referred to in this description are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### EXAMPLES

Bovine vitreous after homogenization was used to model human disease because it mimicked elevated IOP in humans.

In some examples, stock solutions of colistin, polymyxin, and adefovir were prepared in water and then diluted to the desired concentration in PBS to 1% water, with controls were prepared the same way. Once it was added to BVH, the final water concentration was 0.5%.

Bivalirudin, cetylpyridinium, chlorpromazine, boceprevir, levetiracetam, and rapastinel were first prepared in DMSO and diluted in PBS to 1% DMSO. Once added to BVH, the final DMSO concentration was 0.5%.

For some EC50 measurements, the BVH samples were centrifuged at 10 Kg and reconstituted to 25% BVH. For enrichment of BVH, homogenized vitreous humor was centrifuged at 3000 rpm at 4° C. for 30 minutes. The pellet was resuspended with PBS and concentrated by 2.5X fold. For treatment of BVH with compound, 25 ul of enriched BVH was added to 25 ul of Compound and incubated for 1 hour in a thermocycler at 37° C., then 5 ul of 5 mM CFSE was added, and the sample incubated in a thermocycler at 37° C. for 30 minutes.

EC50 was estimated by plotting the logarithmic functions of the micromolar concentration of the drug in the x axis against the percent of maximal response in the y axis. The maximal response was taken as the value of the response for the highest drug concentration. The response was calculated by taking the absolute difference between the control and test value for each concentration.

In some examples, 5 mM CFSE fluorophore was added for imaging a chip.

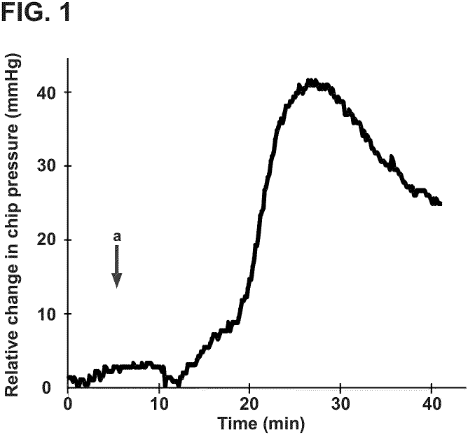

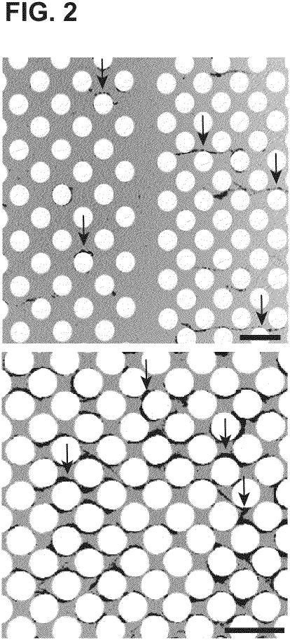

Example 1. Isolation of extracellular matrix bodies in a microfluidic device. FIG. 1 shows that aqueous humor from a patient with primary open angle glaucoma increased the pressure in the microfluidic device. FIG. 1 shows the relative amount of pressure (mm Hg) change within a microfluidic model trabecular meshwork when infused with human aqueous humor obtained from a patient with severe primary open angle glaucoma. The microfluidic channel flow rate was held constant at 2 pl per minute, and the baseline system pressure was measured using an external pressure sensor. The human aqueous humor sample was injected at timepoint denoted by an arrow and the letter “a.” The pressure steadily rises to a maximum of about 41 mm Hg at 27 minutes. FIG. 1 shows that aqueous humor from patients diagnosed with POAG increased the pressure in the device. FIG. 2 (top) shows a confocal photomicrograph of a microfluidic chip after isolating EMB from human aqueous humor from a patient with primary open angle glaucoma, at the end of the experiment shown in FIG. 1. The protein content in the aqueous humor was labeled with a fluorescent marker, carboxyfluorescein succinimidyl ester (CFSE, marked with black arrows in FIG. 2). The circles are pillars in the restriction channel. FIG. 2 (lower) shows EMB isolated in the microfluid channels trapped between pillars (arrowheads). FIG. 2 shows that EMB isolated in the chip increased the pressure in this microfluidic glaucoma model.

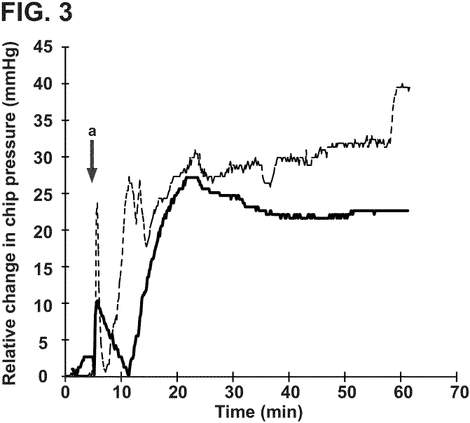

Example 2. Detection and reduction of intraocular pressure (IOP) by an agent in a microfluidic device. FIG. 3 shows that agent colistin sulfate reduced intraocular pressure (IOP) in a human glaucoma model as compared to control. The agent was tested by controlling flow and measuring relative IOP using in a device of this invention. The agent was compared against placebo (buffered saline) by preparing each in aqueous humor from a patient with primary open angle glaucoma and pre-incubating at 37° C. for 24 hours. The timepoint of injection into the device is denoted by an arrow and the letter “a.” Referring to FIG. 3, the IOP for placebo (dashed line) increased greatly after injection of the placebo sample. The IOP rose to a maximum pressure of about 40 mm Hg. To the contrary, the IOP after injection of the agent colistin sulfate in human aqueous humor (solid line) was markedly lower than for placebo, up to about 40% lower, and the difference was sustained. This result showed that the agent colistin sulfate was surprisingly effective to reduce IOP in the human glaucoma model.

Example 3. Dose-response of IOP reducing agents. The dose-response behavior of colistin sulfate on intraocular pressure (IOP) was determined for its use as active agent in treating glaucoma. Colistin sulfate exhibited an EC50 of 0.36 nM for treatment of glaucoma in a bovine vitreous model.

The compound colistin sulfate was tested in bovine vitreous humor (BVH) glaucoma model in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

FIG. 3 shows the dose dependent response curve for the treatment of glaucoma in a bovine vitreous model with the compound colistin sulfate. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 4. Dose-response of IOP reducing agents. The dose-response behavior of cetylpyridinium chloride on intraocular pressure (IOP) was determined for its use as active agent in treating glaucoma. Cetylpyridinium chloride exhibited an EC50 of 0.89 nM for treatment of glaucoma in a bovine vitreous model.

The compound cetylpyridinium chloride was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of glaucoma in a bovine vitreous model. 7 ul of each test solution was injected into the microfluidic chip through a sample injector. Recording of the flow rate and pressure change was continued for 50 additional minutes after the sample injection. The relative change in chip pressure for the entire course of the experiment was obtained.

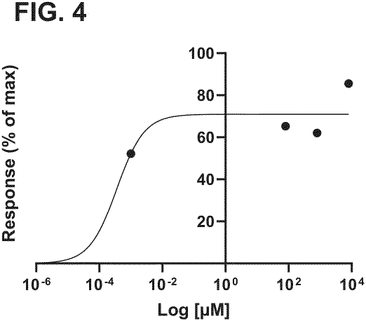

FIG. 4 shows the dose dependent response curve for the treatment of bovine vitreous humor with the compound cetylpyridinium chloride. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 5. Dose-response of IOP reducing agents. The dose-response behavior of polymyxin B sulfate on intraocular pressure (IOP) was determined for its use as active agent in treating glaucoma. Polymyxin B sulfate exhibited an EC50 of 4.3 nM for treatment of glaucoma in a bovine vitreous model.

The compound polymyxin B sulfate was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

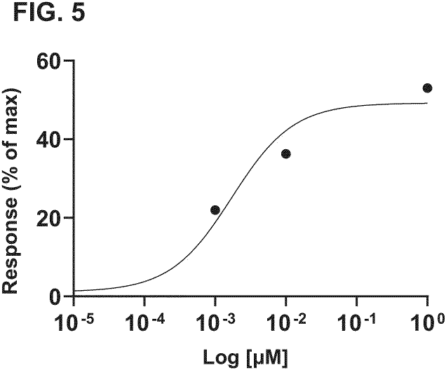

FIG. 5 shows the dose dependent response curve for the treatment of bovine vitreous humor with the compound polymyxin B sulfate. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 6. Dose-response of IOP reducing agents. The dose-response behavior of rapastinel TFA on intraocular pressure (IOP) was determined for its use as active agent in treating glaucoma. Rapastinel TFA exhibited an EC50 of 18 nM for treatment of glaucoma in a bovine vitreous model.

The compound rapastinel TFA was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

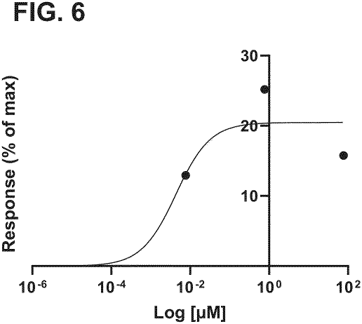

FIG. 6 shows the dose dependent response curve for the treatment of bovine vitreous humor with the compound rapastinel TFA. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 7. Dose-response of IOP reducing agents. The dose-response behavior of adefovir on intraocular pressure (IOP) was determined for its use as active agent in treating glaucoma. Adefovir exhibited an EC50 of 169 nM for treatment of glaucoma in a bovine vitreous model.

The compound adefovir was tested in bovine vitreous humor (BVH) glaucoma model in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

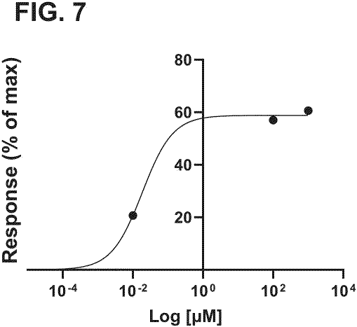

FIG. 7 shows the dose dependent response curve for the treatment of bovine vitreous humor with the compound adefovir. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 8. Dose-response of IOP reducing agents. The dose-response behavior of levetiracetam on intraocular pressure (IOP) was determined for its use as active agent in treating glaucoma. Levetiracetam exhibited an EC50 of 213 nM for treatment of glaucoma in a bovine vitreous model.

The compound levetiracetam was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

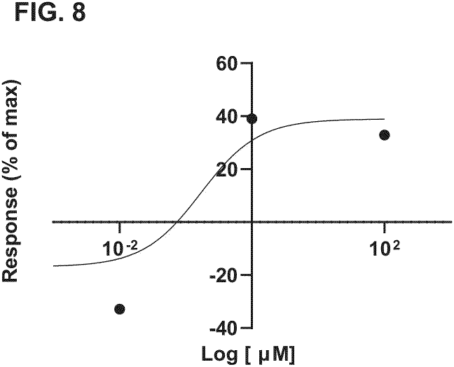

FIG. 8 shows the dose dependent response curve for the treatment of bovine vitreous humor with the compound levetiracetam. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 9. Dose-response of IOP reducing agents. The dose-response behavior of chlorpromazine on intraocular pressure (IOP) was determined for its use as active agent in treating glaucoma. Chlorpromazine exhibited an EC50 of 11,320 nM for treatment of glaucoma in a bovine vitreous model.

The compound chlorpromazine was tested in bovine vitreous humor (BVH) in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

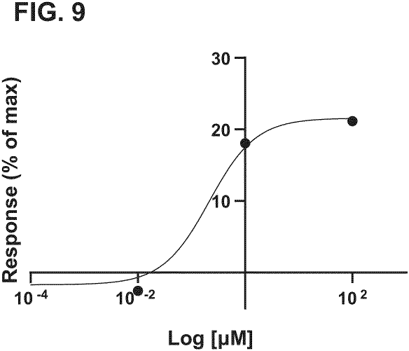

FIG. 9 shows the dose dependent response curve for the treatment of bovine vitreous humor with the compound chlorpromazine. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 10. Dose-response of IOP reducing agents. The dose-response behavior of boceprevir on intraocular pressure (IOP) was determined for its use as active agent in treating glaucoma. Boceprevir exhibited an EC50 of 11,270 nM for treatment of glaucoma in a bovine vitreous model.

The compound boceprevir was tested in bovine vitreous humor (BVH) glaucoma model in a microfluidic chip device. A solution of 25% homogenized BVH in PBS buffer was prepared and diluted with an equal amount of a solution of the compound, so that the total BVH concentration was 12.5%. The sample was vortexed and incubated at 37° C. for 1 hour. A control of either PBS buffer or PBS with 10% ultrapure water or DMSO was used and incubated with BVH under the same conditions.

The test compound-BVH solution was introduced into the reservoir of the microfluidic chip device and flow rate and pressure change were recorded. Various concentrations of the compound were tested for effects on the treatment of bovine vitreous humor. 7 ul of each test solution was injected into the microfluidic chip through a sample injector. Recording of the flow rate and pressure change was continued for 50 additional minutes after the sample injection. The relative change in chip pressure for the entire course of the experiment was obtained.

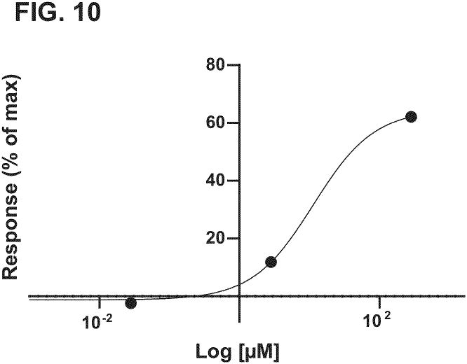

FIG. 10 shows the dose dependent response curve for the treatment of bovine vitreous humor with the compound boceprevir. The EC50 value was taken as the point on the x-axis at which the logarithmic function of the micromolar concentration of the compound produced half-maximal response. The logarithmic function of the micromolar concentration of the drug was plotted on the x axis against the percent of maximal response on the y axis. Maximal response was obtained by taking the value of the response for the highest drug concentration. The response was calculated by taking the absolute difference between the control and test value for each concentration.

Example 11. An active agent for use in treating glaucoma can be polymyxin B.

A solution of the agent compound was prepared by weighing out the compound in a microcentrifuge tube and dissolving the solid material in lx PBS buffer at pH 7.2. For a compound that was less soluble in water, a stock solution was prepared in ethanol or DMSO, and then diluted ten-fold to achieve the final concentration with a 10% ethanol or DMSO vehicle. Heat (37° C.) and vortex mixing were applied to the solution of the compound to facilitate dissolution.

The concentration of Polymyxin B sulfate was 10 mg/ml.

To determine the effect of the compound on intraocular pressure (IOP), the compound was tested in bovine vitreous humor (BVH) glaucoma model in the microfluidic chip device.

A solution of 25% homogenized bovine vitreous humor (BVH) was prepared by diluting 100% homogenized BVH with PBS buffer. 50 uL BVH was aliquoted into 0.5 mL PCR tubes. 50 uL of the solution of the compound was added to the BVH, bringing the BVH total concentration to 12.5%. The sample was briefly vortexed and then incubated at 37° C. overnight. For a control experiment, 50 uL of either PBS buffer or PBS with 10% ethanol or DMSO were prepared and incubated with 25% BVH in the same conditions.

The test BVH solution was introduced into the reservoir of the device. A fluidic probe was attached to the inlet of the microfluidic chip and a flow rate of 2ul/min was established with PBS as the source fluid. Once fluid began exiting from the outlet of the chip and a steady flow of 2 ul/min was achieved, the flow rate and the pressure change within the microfluidic chip were recorded. Baseline flow rate and pressure readings were recorded for 5 minutes, after which 7 ul of the test BVH solution was injected into the chip through a sample injector. Recording of the flow rate and pressure change was continued for 50 additional minutes after the sample injection. Recording was stopped after 55 minutes. The relative change in chip pressure for the entire course of the experiment was plotted on a graph.

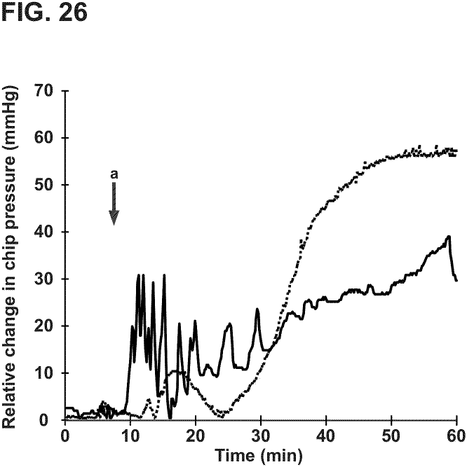

FIG. 26 shows that agent polymyxin B reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 26, the IOP for placebo (dashed line) increased greatly after injection of the placebo sample. The IOP rose steadily to a maximum pressure of about 250 mmHg. To the contrary, the IOP after injection of the agent polymyxin B (solid line) was 78% lower than for placebo, and the difference was sustained. This result showed that the agent polymyxin B was surprisingly effective to reduce IOP in the glaucoma model.

Example 12. An active agent for use in treating glaucoma can be neomycin.

A solution of the agent compound was prepared by weighing out the compound in a microcentrifuge tube and dissolving the solid material in lx PBS buffer at pH 7.2. For a compound that was less soluble in water, a stock solution was prepared in ethanol or DMSO, and then diluted ten-fold to achieve the final concentration with a 10% ethanol or DMSO vehicle. Heat (37° C.) and vortex mixing were applied to the solution of the compound to facilitate dissolution.

The concentration of neomycin sulfate was 35 mg/ml.

To determine the effect of the compound on intraocular pressure (IOP), the compound was tested in bovine vitreous humor (BVH) glaucoma model in the microfluidic chip device.

A solution of 25% homogenized bovine vitreous humor (BVH) was prepared by diluting 100% homogenized BVH with PBS buffer. 50 uL BVH was aliquoted into 0.5 mL PCR tubes. 50 uL of the solution of the compound was added to the BVH, bringing the BVH total concentration to 12.5%. The sample was briefly vortexed and then incubated at 37° C. overnight. For a control experiment, 50 uL of either PBS buffer or PBS with 10% ethanol or DMSO were prepared and incubated with 25% BVH in the same conditions.

The test BVH solution was introduced into the reservoir of the device. A fluidic probe was attached to the inlet of the microfluidic chip and a flow rate of 2 ul/min was established with PBS as the source fluid. Once fluid began exiting from the outlet of the chip and a steady flow of 2 ul/min was achieved, the flow rate and the pressure change within the microfluidic chip were recorded. Baseline flow rate and pressure readings were recorded for 5 minutes, after which 7 ul of the test BVH solution was injected into the chip through a sample injector. Recording of the flow rate and pressure change was continued for 50 additional minutes after the sample injection. Recording was stopped after 55 minutes. The relative change in chip pressure for the entire course of the experiment was plotted on a graph.

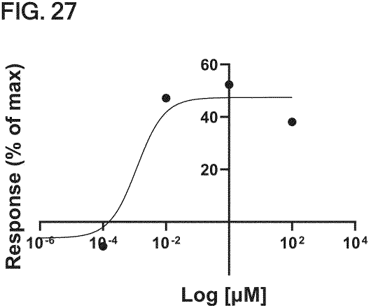

FIG. 27 shows that agent neomycin reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 27, the IOP for placebo (dashed line) increased greatly after injection of the placebo sample. The IOP rose steadily to a maximum pressure of about 64 mmHg. To the contrary, the IOP after injection of the agent neomycin (solid line) was 72% lower than for placebo, and the difference was sustained. This result showed that the agent neomycin was surprisingly effective to reduce IOP in the glaucoma model.

Example 13. FIG. 28 shows that agent colistin sulfate reduced intraocular pressure (IOP) in a bovine glaucoma model as compared to control. The agent was tested by controlling flow and measuring relative IOP using in a device of this invention. The agent was compared against placebo (buffered saline) by preparing each in bovine aqueous humor (BVH) and pre-incubating at 37° C. for 24 hours. The timepoint of injection into the device is denoted by an arrow and the letter “a.” Referring to FIG. 28, the IOP for placebo (dashed line) increased greatly after injection of the placebo sample. The IOP rose to a maximum pressure of about 65 mm Hg. To the contrary, the IOP after injection of the agent colistin sulfate in BVH (solid line) was markedly lower than for placebo, up to about 97% lower, and the difference was sustained. This result showed that the agent colistin sulfate was surprisingly effective to reduce IOP in the glaucoma model.

Example 14. Sodium dodecyl sulfate was a negative control for intraocular pressure (IOP) in a glaucoma model.

A solution was prepared by weighing out the compound in a microcentrifuge tube and dissolving the solid material in lx PBS buffer at pH 7.2. For a compound that was less soluble in water, a stock solution was prepared in ethanol or DMSO, and then diluted ten-fold to achieve the final concentration with a 10% ethanol or DMSO vehicle. Heat (37° C.) and vortex mixing were applied to the solution of the compound to facilitate dissolution.

The concentration of sodium dodecyl sulfate was 24 mg/ml.

To determine the effect of the compound on intraocular pressure (IOP), the compound was tested in bovine vitreous humor (BVH) glaucoma model in the microfluidic chip device.

A solution of 25% homogenized bovine vitreous humor (BVH) was prepared by diluting 100% homogenized BVH with PBS buffer. 50 uL BVH was aliquoted into 0.5 mL PCR tubes. 50 uL of the solution of the compound was added to the BVH, bringing the BVH total concentration to 12.5%. The sample was briefly vortexed and then incubated at 37° C. overnight. For a control experiment, 50 uL of either PBS buffer or PBS with 10% ethanol or DMSO were prepared and incubated with 25% BVH in the same conditions.

The test BVH solution was introduced into the reservoir of the device. A fluidic probe was attached to the inlet of the microfluidic chip and a flow rate of 2 ul/min was established with PBS as the source fluid. Once fluid began exiting from the outlet of the chip and a steady flow of 2 ul/min was achieved, the flow rate and the pressure change within the microfluidic chip were recorded. Baseline flow rate and pressure readings were recorded for 5 minutes, after which 7 ul of the test BVH solution was injected into the chip through a sample injector. Recording of the flow rate and pressure change was continued for 50 additional minutes after the sample injection. Recording was stopped after 55 minutes. The relative change in chip pressure for the entire course of the experiment was plotted on a graph.

FIG. 29 shows that compound sodium dodecyl sulfate was a negative control for intraocular pressure (IOP) in a glaucoma model. The compound was tested by controlling flow and measuring relative IOP using in a device of this invention. The compound was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 29, the IOP for placebo (dashed line) increased greatly after injection of the placebo sample. The IOP rose steadily to a maximum pressure of about 60 mm Hg. However, the IOP after injection of sodium dodecyl sulfate (solid line) was significantly higher than for placebo. This result showed that sodium dodecyl sulfate was a negative control that did not reduce IOP in the glaucoma model.

Example 15. An active agent for use in treating glaucoma can be cetylpyridinium.

A solution of the agent compound was prepared by weighing out the compound in a microcentrifuge tube and dissolving the solid material in lx PBS buffer at pH 7.2. For a compound that was less soluble in water, a stock solution was prepared in ethanol or DMSO, and then diluted ten-fold to achieve the final concentration with a 10% ethanol or DMSO vehicle. Heat (37° C.) and vortex mixing were applied to the solution of the compound to facilitate dissolution.

The concentration of cetylpyridinium chloride was 43 mg/ml.

To determine the effect of the compound on intraocular pressure (IOP), the compound was tested in bovine vitreous humor (BVH) glaucoma model in the microfluidic chip device.

A solution of 25% homogenized bovine vitreous humor (BVH) was prepared by diluting 100% homogenized BVH with PBS buffer. 50 uL BVH was aliquoted into 0.5 mL PCR tubes. 50 uL of the solution of the compound was added to the BVH, bringing the BVH total concentration to 12.5%. The sample was briefly vortexed and then incubated at 37° C. overnight. For a control experiment, 50 uL of either PBS buffer or PBS with 10% ethanol or DMSO were prepared and incubated with 25% BVH in the same conditions.

The test BVH solution was introduced into the reservoir of the device. A fluidic probe was attached to the inlet of the microfluidic chip and a flow rate of 2 ul/min was established with PBS as the source fluid. Once fluid began exiting from the outlet of the chip and a steady flow of 2 ul/min was achieved, the flow rate and the pressure change within the microfluidic chip were recorded. Baseline flow rate and pressure readings were recorded for 5 minutes, after which 7 ul of the test BVH solution was injected into the chip through a sample injector. Recording of the flow rate and pressure change was continued for 50 additional minutes after the sample injection. Recording was stopped after 55 minutes. The relative change in chip pressure for the entire course of the experiment was plotted on a graph.

FIG. 30 shows that agent cetylpyridinium chloride reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 30, the IOP for placebo (dashed line) increased greatly after injection of the placebo sample. The IOP rose steadily to a maximum pressure of about 64 mm Hg. To the contrary, the IOP after injection of the agent cetylpyridinium chloride-BVH sample (solid line) was markedly lower than for placebo, up to nearly 100% lower, and the difference was sustained. This result showed that the agent cetylpyridinium chloride was surprisingly effective to reduce IOP in the glaucoma model.

Example 16. FIG. 31 shows that agent chlorpromazine reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 31, the IOP for placebo (dashed line) increased greatly after injection of the placebo sample. The IOP rose steadily to a maximum pressure of about 64 mm Hg. To the contrary, the IOP after injection of the agent chlorpromazine-BVH sample (solid line) was markedly lower than for placebo, up to about 81% lower, and the difference was sustained. This result showed that the agent chlorpromazine was surprisingly effective to reduce IOP in the glaucoma model.

Example 17. An active agent for use in treating glaucoma can be heparin.

A solution of the agent compound was prepared by weighing out the compound in a microcentrifuge tube and dissolving the solid material in lx PBS buffer at pH 7.2. For a compound that was less soluble in water, a stock solution was prepared in ethanol or DMSO, and then diluted ten-fold to achieve the final concentration with a 10% ethanol or DMSO vehicle. Heat (37° C.) and vortex mixing were applied to the solution of the compound to facilitate dissolution.

The concentration of heparin sodium was 10 mg/ml.

To determine the effect of the compound on intraocular pressure (IOP), the compound was tested in bovine vitreous humor (BVH) in the microfluidic chip device.

A solution of 25% homogenized bovine vitreous humor (BVH) was prepared by diluting 100% homogenized BVH with PBS buffer. 50 uL BVH was aliquoted into 0.5 mL PCR tubes. 50 uL of the solution of the compound was added to the BVH, bringing the BVH total concentration to 12.5%. The sample was briefly vortexed and then incubated at 37° C. overnight. For a control experiment, 50 uL of either PBS buffer or PBS with 10% ethanol or DMSO were prepared and incubated with 25% BVH in the same conditions.

The test BVH solution was introduced into the reservoir of the device. A fluidic probe was attached to the inlet of the microfluidic chip and a flow rate of 2 ul/min was established with PBS as the source fluid. Once fluid began exiting from the outlet of the chip and a steady flow of 2 ul/min was achieved, the flow rate and the pressure change within the microfluidic chip were recorded. Baseline flow rate and pressure readings were recorded for 5 minutes, after which 7 ul of the test BVH solution was injected into the chip through a sample injector. Recording of the flow rate and pressure change was continued for 50 additional minutes after the sample injection. Recording was stopped after 55 minutes. The relative change in chip pressure for the entire course of the experiment was plotted on a graph.

FIG. 32 shows that agent heparin sodium reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 32, the IOP for placebo (dashed line) increased greatly after injection of the placebo sample. The IOP rose steadily to a maximum pressure of about 67 mmHg. To the contrary, the IOP after injection of the agent heparin sodium (solid line) was 32% lower than for placebo, and the difference was sustained. This result showed that the agent heparin sodium was surprisingly effective to reduce IOP in the glaucoma model.

Example 18. FIG. 33 shows that agent adefovir dipivoxil reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 33, the IOP for placebo (dashed line) increased greatly after injection of the placebo sample. The IOP rose steadily to a maximum pressure of about 112 mmHg. To the contrary, the IOP after injection of the agent adefovir dipivoxil (solid line) was up to 73% lower than for placebo, and the difference was sustained. This result showed that the agent adefovir dipivoxil was surprisingly effective to reduce IOP in the glaucoma model.

Example 19. FIG. 34 shows that agent triflupromazine reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 34, the IOP for placebo (dashed line) increased greatly after injection of the placebo sample. The IOP rose steadily to a maximum pressure of about 112 mm Hg. To the contrary, the IOP after injection of the agent triflupromazine (solid line) was up to 40% lower than for placebo, and the difference was sustained. This result showed that the agent triflupromazine was surprisingly effective to reduce IOP in the glaucoma model.

Example 20. FIG. 35 shows that agent bacitracin zinc reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 35, the IOP for placebo (dashed line) increased greatly after injection of the placebo sample. The IOP rose steadily to a maximum pressure of about 113 mm Hg. To the contrary, the IOP after injection of the agent bacitracin zinc (solid line) was up to 58% lower than for placebo, and the difference was sustained. This result showed that the agent bacitracin zinc was surprisingly effective to reduce IOP in the glaucoma model.

Example 21. FIG. 36 shows that agent levetiracetam reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 36, the IOP for placebo (dashed line) increased greatly after injection of the placebo sample. The IOP rose steadily to a maximum pressure of about 55 mm Hg. To the contrary, the IOP after injection of the agent levetiracetam (solid line) was up to 62% lower than for placebo, and the difference was sustained. This result showed that the agent levetiracetam was surprisingly effective to reduce IOP in the glaucoma model.

Example 22. FIG. 37 shows that compound ombitasvir was a negative control for intraocular pressure (IOP) in a glaucoma model. The compound was tested by controlling flow and measuring relative IOP using in a device of this invention. The compound was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 37, the IOP for placebo (dashed line) increased greatly after injection of the placebo sample. The IOP rose steadily to a maximum pressure of about 110 mm Hg. However, the IOP after injection of ombitasvir (solid line) was significantly higher than for placebo. This result showed that ombitasvir was a negative control that did not reduce IOP in the glaucoma model.

Example 23. FIG. 38 shows that agent boceprevir reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 38, the IOP for placebo (dashed line) increased greatly after injection of the placebo sample. The IOP rose steadily to a maximum pressure of about 112 mm Hg. To the contrary, the IOP after injection of the agent boceprevir (solid line) was up to 67% lower than for placebo, and the difference was sustained. This result showed that the agent boceprevir was surprisingly effective to reduce IOP in the glaucoma model.

Example 24. FIG. 39 shows that agent rapastinel TFA reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 39, the IOP for placebo (dashed line) increased greatly after injection of the placebo sample. The IOP rose steadily to a maximum pressure of about 57 mm Hg. To the contrary, the IOP after injection of the agent rapastinel TFA (solid line) was up to 82% lower than for placebo, and the difference was sustained. This result showed that the agent rapastinel TFA was surprisingly effective to reduce IOP in the glaucoma model.

Example 25. FIG. 40 shows that agent pramiracetam reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 40, the IOP for placebo (dashed line) increased greatly after injection of the placebo sample. The IOP rose steadily to a maximum pressure of about 57 mm Hg. To the contrary, the IOP after injection of the agent pramiracetam (solid line) was up to 44% lower than for placebo, and the difference was sustained. This result showed that the agent pramiracetam was surprisingly effective to reduce IOP in the glaucoma model.

Example 26. FIG. 41 shows that agent bivalirudin reduced intraocular pressure (IOP) in a glaucoma model as compared to control. The agent was tested by controlling flow and measuring relative IOP using in a device of this invention. The agent was compared against placebo (buffered saline) by preparing each in bovine vitreous humor (BVH) and pre-incubating at 37° C. for 24 hours. The timepoint of injection into the device is denoted by an arrow and the letter “a.” Referring to FIG. 41, the IOP for placebo (dashed line) increased greatly after injection of the placebo sample. The IOP rose steadily to a maximum pressure of about 112 mm Hg. To the contrary, the IOP after injection of the agent bivalirudin (solid line) was up to 91% lower than for placebo, and the difference was sustained. This result showed that the agent bivalirudin was surprisingly effective to reduce IOP in the glaucoma model.

1-197. (canceled)

198. A pharmaceutical composition for ophthalmic use comprising a cyclic peptide active agent.

199. The composition of claim 198, wherein the cyclic peptide is a cyclic hepapeptide with a tripeptide side branch.

200. The composition of claim 198, wherein the active agent has Formula XV 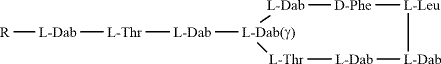
wherein R is selected from alkyl, cycloalkyl, aminoalkyl, alkenyl, alkynyl, alkanoyl, alkenoyl, wherein Dab is a diaminobutanoic acid monomer, and pharmaceutically-acceptable prodrugs, esters and salts thereof.

201. The composition of claim 200, wherein R is 6-methyloctanoyl (B₁), 6-methylheptanoyl (B₂), octanoyl (B₃), heptanoyl (B₄), and pharmaceutically-acceptable prodrugs, esters and salts thereof.

202. The composition of claim 200, wherein
R is selected from alkyl, cycloalkyl, aminoalkyl, alkenyl, alkynyl, alkanoyl, alkenoyl;
and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

203. The composition of claim 198, wherein the active agent has Formula XVI 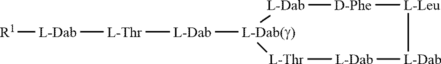
wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy;
and pharmaceutically-acceptable prodrugs, esters and salts thereof.

204. The composition of claim 203, wherein
wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy;
and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

205. The composition of claim 203, wherein
R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

206. The composition of claim 203, wherein
wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof;
preferably excluding polymyxin, polymyxin B for use in treating glaucoma;
more preferably excluding polymyxin, polymyxin B and all pharmaceutically acceptable prodrugs, esters and salts thereof for use in treating glaucoma;
even more preferably excluding polymyxin, polymyxin B and all pharmaceutically acceptable prodrugs, esters and salts thereof for any use.

207. The composition of claim 198, wherein the active agent has Formula XVII 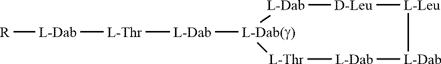
wherein R is selected from alkyl, cycloalkyl, aminoalkyl, alkenyl, alkynyl, alkanoyl, alkenoyl, and pharmaceutically-acceptable prodrugs, esters and salts thereof.

208. The composition of claim 207, wherein R is selected from alkyl, cycloalkyl, aminoalkyl, alkenyl, alkynyl, alkanoyl, alkenoyl, and pharmaceutically-acceptable prodrugs, esters and salts thereof.

209. The composition of claim 198, wherein the active agent has Formula XVIII 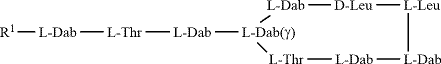
wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

210. The composition of claim 209, wherein R¹ is a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid, or a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

211. The composition of claim 209, wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

212. The composition of claim 209, wherein R¹ is a substituted or unsubstituted C(12-22)alkyl, C(6-12)cycloalkyl, C(6-12)cycloalkyl-C(12-22)alkyl, C(12-22)alkenyl, C(12-22)alkynyl, C(12-22)alkoxy, C(6-12)alkoxy-C(12-22)alkyl, C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

213. The composition of claim 209, wherein R¹ is a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

214. The composition of claim 209, wherein R¹ is a substituted or unsubstituted C(12-22)alkanoyl, C(6-12)cycloalkyl-C(12-22)alkanoyl, C(12-22)alkenoyl, or C(12-22)alkanoyloxy, and pharmaceutically-acceptable prodrugs, esters and salts thereof.

215. The composition of claim 198, wherein the active agent has Formula XIX wherein 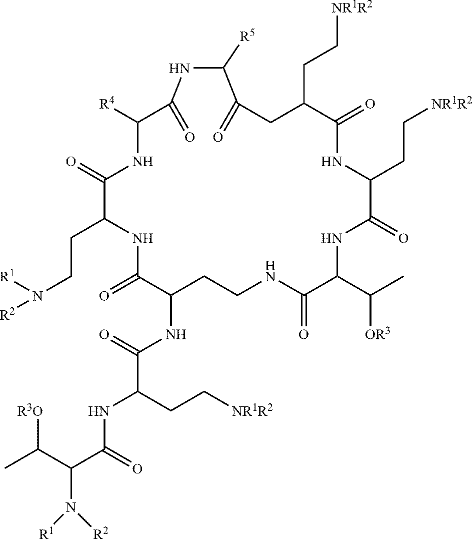
R¹, R² are independently selected from H, alkyl, cycloalkyl aminoalkyl, hydroxyalkyl, carboxylalkyl, aryl;
R³ is selected from H, alkyl, aminoalkyl, hydroxyalkyl, carboxylalkyl;
R⁴ is selected from H, alkyl, cycloalkyl, aminoalkyl, hydroxyalkyl, carboxylalkyl, benzyl, aryl, aralkyl, cycloalkyl-alkyl;
R⁵ is selected from H, alkyl, cycloalkyl, aminoalkyl, hydroxyalkyl, carboxylalkyl, aryl;
and pharmaceutically-acceptable prodrugs, esters and salts thereof.

216. The composition of claim 198, wherein the active agent has Formula XX 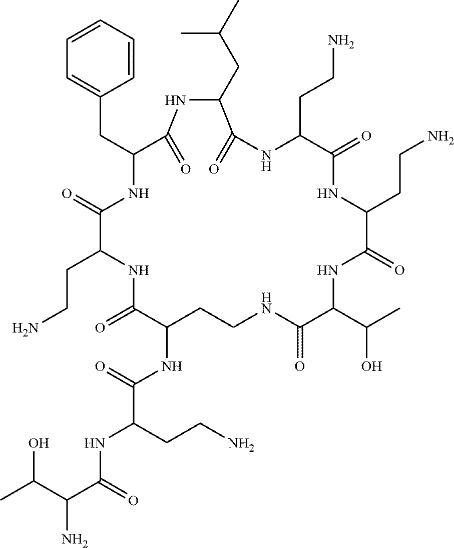 [SMILES: CC(C)CC1NC(=O)C(Cc2ccccc2)NC(=O)C(CCN)NC(=O)C(NC(=O)C(CCN)NC(=O)C(N)C(C)O)CCNC(=O)C(C(C)O)NC(=O)C(CCN)NC(=O)C(CCN)NC1=O]
and pharmaceutically-acceptable prodrugs, esters and salts thereof.

217. The composition of claim 198, wherein the active agent has Formula XXI 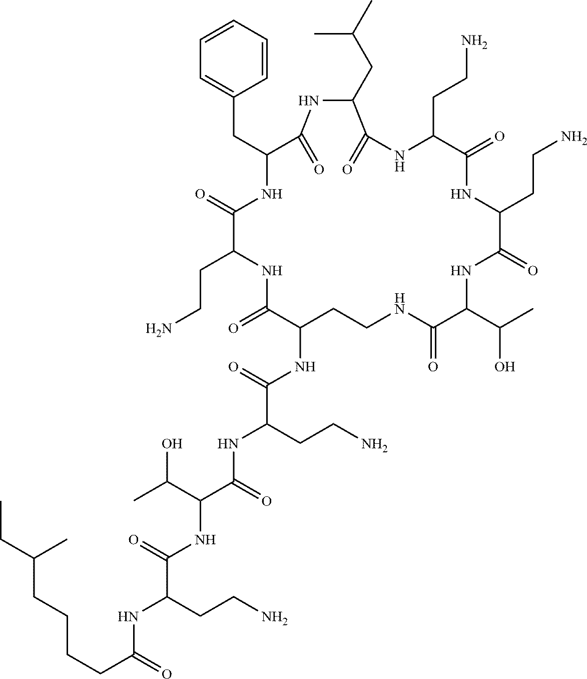 [SMILES: CCC(C)CCCCC(=O)NC(CCN)C(=O)NC(C(=O)NC(CCN)C(=O)NC1CCNC(=O)C(C(C)O)NC(=O)C(CCN)NC(=O)C(CCN)NC(=O)C(CC(C)C)NC(=O)C(Cc2ccccc2)NC(=O)C(CCN)NC1=O)C(C)O]
and pharmaceutically-acceptable prodrugs, esters and salts thereof.

218. The composition of claim 198, wherein the active agent has Formula XXII 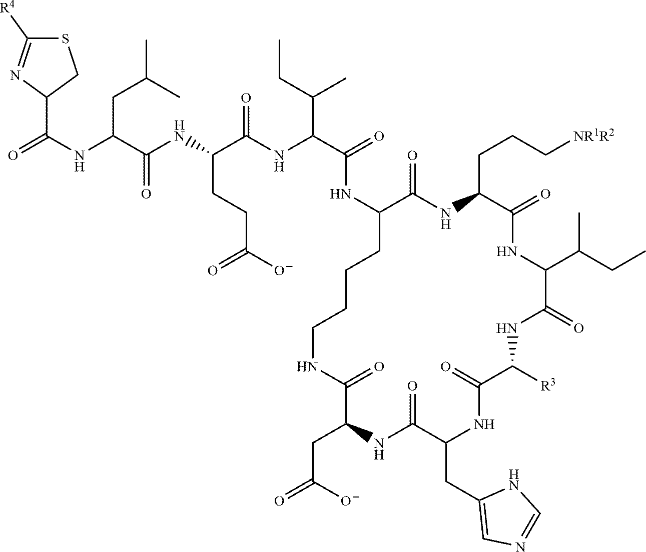
wherein R¹, R² are independently selected from H, alkyl, cycloalkyl, aminoalkyl, hydroxyalkyl, carboxylalkyl, aryl;
R³ is selected from H, alkyl, cycloalkyl, aryl, benzyl, arylalkyl;
R⁴ is selected from H, alkyl, cycloalkyl, aryl, aminoalkyl, arylalkyl;
and pharmaceutically-acceptable prodrugs, esters and salts thereof.

219. The composition of claim 218, wherein the active agent is bacitracin A, and pharmaceutically-acceptable prodrugs, esters and salts thereof.


SEQUENCE LISTING (2 sequences)

SEQ ID NO: 1 - protein, 13 aa, synthetic construct
  REGION 1..13: note = Description of Artificial Sequence: Syntheticpeptide
NGDFEEIPEEYLA

SEQ ID NO: 2 - protein, 4 aa, synthetic construct
  REGION 1..4: note = Description of Artificial Sequence: Syntheticpeptide
TPPT